(19) United States
(12) Patent Application Publication
(10) Pub. No.: US 2022/0160872 A1
(43) Pub. Date: May 26, 2022

(54) Drug Adsorbed Highly Porous Activated Carbon for Enhanced Drug Delivery

(71) Applicant: THE BOARD OF TRUSTEES OF THE UNIVERSITY OF ILLINOIS, Urbana, IL (US)

(72) Inventors: Tejabhiram Yadavalli, Chicago, IL (US); Deepak Shukla, Chicago, IL (US)

(21) Appl. No.: 17/601,449

(22) PCT Filed: Apr. 8, 2020
(86) PCT No.: PCT/US20/27299

§ 371 Date: Oct. 5, 2021

Related U.S. Application Data
(60) Provisional application No. 62/831,425, filed on Apr. 9, 2019.

(51) Int. Cl. A61K 47/02; A61K 45/06; A61K 47/69; A61K 9/00; A61P 31/22; A61K 31/522

(52) U.S. Cl. CPC A61K 47/02; A61K 45/06; A61K 47/6929; B82Y 5/00; A61K 9/0034; A61P 31/22; A61K 31/522; A61K 9/0004

(57) ABSTRACT

The present disclosure relates to compositions and drug delivery systems comprising highly porous activated carbon (HPAC) and a therapeutic agent and methods of treating a disorder using these compositions and drug delivery systems. The present disclosure also provides for methods of treating viral infections with HPAC or therapeutic agents adsorbed within HPAC and methods of eliciting an immune response comprising administering a live, live-attenuated virus or virion adsorbed to HPAC.

### CROSS REFERENCE TO RELATED APPLICATIONS

This application claims the benefit of U.S. Provisional Patent Application No. 62/831,425, filed on Apr. 9, 2019, which is incorporated herein by reference in its entirety.

### STATEMENT REGARDING FEDERALLY FUNDED RESEARCH

The present disclosure was made with government support under grant numbers RO1 EY024710, RO1 AI139768, and RO1 EY029426 awarded by the National Institutes of Health). The government has certain rights in the disclosure

### FIELD OF THE DISCLOSURE

The present disclosure relates to compositions and drug delivery systems comprising highly porous activated carbon (HPAC) and a therapeutic agent and methods of treating a disorder using these compositions and drug delivery systems. The present disclosure also provides for methods of treating viral infections with HPAC or therapeutic agents adsorbed within HPAC and methods of eliciting an immune response comprising administering a live, live-attenuated virus or virion adsorbed to HPAC.

### BACKGROUND OF THE DISCLOSURE

The development of a novel therapeutic or prophylactic drug against one of the world's most common pathogens, herpes simplex virus (HSV), has been under extensive investigation in recent years with hopes of ultimately finding a more effective way to treat the infection. Herpesviridae is a family of double-stranded DNA-enveloped viruses that encompasses some of the most widespread viruses among the human population. Among these is HSV which has two serotypes: HSV-1 and HSV-2. HSV-1 is the leading cause of fever blisters and other mucocutaneous lesions including those in the cornea, which leads to vision loss or blindness. HSV-2 is associated with genital lesions and warts and both viruses are capable of causing central nervous system diseases (e.g. encephalitis and meningitis) leading to death in untreated patients. Strikingly, it is estimated that over a third of the world population currently suffers from latent HSV infection. Additionally, HSV infection has been implicated with increased risk of contracting other sexually transmitted diseases, such as human immunodeficiency virus (HIV) and human papilloma virus (HPV), and as a result has garnered much attention in the recent years.

The most common mode of treatment for HSV infection includes daily dosing of orally administered Acyclovir (ACV). While this treatment is effective in most cases, various reports have shown that long term systemic use of ACV results in resistance against the drug and may cause renal injury. Sustained delivery of pharmacologically active ACV is also an issue and drug efficacy are often compromised due to this problem. While topical medications in the form of ganciclovir (ACV analogue) gels and trifluridine (TFT) have been used for HSV infection, they have shown low retention time on corneal and vaginal surfaces and require repeated dosing (6-10 times daily). To provide a better alternative, experimental treatments such as antibodies, peptides, small molecule inhibitors, aptamers and nanoparticles have been tested. While most demonstrated effective antiviral activity against HSV both in-vitro and in-vivo, they all also suffer from low retention time and delivery issues on mucosal surfaces. A simple yet effective way of resolving this problem is to develop a topical treatment that protects the site of infection while providing antiviral relief for an extended period.

### SUMMARY OF THE DISCLOSURE

The studies provided herein demonstrate that charcoal in its surface-active form could trap the virions and provide antiviral effects. In addition, synergy studies with Acyclovir (ACV) revealed that ACV may be adsorbed and/or is encapsulated within HPAC resulting in a sustained release of ACV that generated a stronger therapeutic effect. This novel antiviral drug delivery system is referred to herein as “drug encapsulated carbon” (DECON). DECON provided better therapeutic efficacy via sustained release of the antiviral drug, e.g. ACV, and given its non-toxic nature and kidney function related benefits, this drug delivery system circumvents the renal toxicity issues associated with ACV. As a virus-trapping compound activated charcoal, is also contemplated to be a platform for mucosal vaccine delivery of live, live-attenuated, or fixed virions.

The disclosure provides for compositions comprising drug encapsulated carbon for delivery many types of drugs for the treatment of human and non-human diseases.

The disclosure provides for compositions comprising highly porous activated carbon (HPAC) and a therapeutic agent. In any of the disclosed compositions, the HPAC is a microporous carbon. In some embodiments, the HPAC in the disclosed compositions has a pore size ranging from about 10 Å to about 20 Å, or has a cumulative pore volume ranging from about 25 cc/g to about 0.75 cc/g or has a total pore volume ranging from about 500 m²/g to about 3000 m²/g. In additional embodiments, the HPAC has a particle size distribution ranging from about 10 nm to about 500 μM.

In any of the provided compositions, the therapeutic agent can by any known in the art for treating a human or non-human subject. In some embodiments, the therapeutic agent is an anti-viral agent, antibacterial agent, growth factor, cytokine, anticancer and/or cytotoxic agent, analgesic, anti-hypertension drug, anti-allergenic agent, an anti-seizure compound, non-steroidal anti-inflammatory drug, an antibiotic, growth hormone, parathyroid hormone, insulin, interferons, chemotherapeutic agent, analgesic, immune suppression agent, antidepressant, antidiabetic agent, anti-parasitic, antidiarrheal agent, antimigraine agents, antipsychotic, antiparkinsonian agent, anxiolytic, or hypotensive agent.

In an exemplary embodiment, the provided composition comprises HPAC and an anti-viral agent. For example, the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine and sofobuvir.

In another embodiment, the provided composition comprises HPAV and a live virus, live-attenuated virus or a fixed virion. For example, the virus, live-attenuated virus or fixed viron is Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus. In some embodiments, the provided compositions further comprise an adjuvant.

The disclosure also provides for any of the disclosed compositions further comprising a pharmaceutically acceptable carrier.

The disclosure provides for a drug delivery system comprising a therapeutic agent adsorbed to HPAC. In any of the disclosed compositions, the HPAC is a microporous carbon. In some embodiments, the HPAC in the disclosed compositions has a pore size ranging from about 10 Å to about 20 Å, or has a cumulative pore volume ranging from about 25 cc/g to about 0.75 cc/g or has a total pore volume ranging from about 500 m²/g to about 3000 m²/g. In additional embodiments, the HPAC has a particle size distribution ranging from about 10 nm to about 500 μM.

In some embodiments, the drug delivery system is administered by parenteral, nasal, oral, pulmonary, topical, vaginal, or rectal delivery. In addition, any of the disclosed drug delivery systems delivers the therapeutic agent by sustained release. In some embodiments, any of the disclosed drug delivery systems prophylactically deliver the therapeutic agent, wherein prophylactic delivery refers to administration of the therapeutic agent prior to infection, or prior to the onset of symptoms of the disorder or condition. In other embodiments, any of the drug delivery systems therapeutically deliver the therapeutic agent, wherein therapeutic delivery refers to administration of the therapeutic agent after or during active infection or during onset of the disorder or condition, e.g. while symptoms are observed for the condition or disorder.

For example, for viral infection, prophylactic delivery refer to administration of HPAC, the therapeutic agent, e.g. anti-viral agent, or drug delivery system prior to infection in order to prevent or delay infection. In some aspects, prophylactic delivery prevents virus from entering into a cell, or inhibiting viral spread from an infected cell to an uninfected cell.

In some embodiments, for viral infection, therapeutic delivery refers to administration of HPAC, the therapeutic agent, e.g. anti-viral agent, or drug delivery system after virus infection or during active viral infection or during onset of symptoms caused by viral infection. Therapeutic delivery includes neutralization treatment. In some aspects, therapeutic delivery includes one or more of reducing or preventing virus replications, reducing viral load, reducing or preventing viral spread, e.g. inhibiting syncytia formation, and reducing or preventing viral entry into a cell.

In any of the disclosed drug delivery systems, the therapeutic agent adsorbed to the HPAC is an anti-viral agent, antibacterial agent, growth factor, cytokine, anticancer and/or cytotoxic agent, analgesic, anti-hypertension drug, anti-allergenic agent, an anti-seizure compound, non-steroidal anti-inflammatory drug, an antibiotic, growth hormone, parathyroid hormone, insulin, interferons, chemotherapeutic agent, analgesic, immune suppression agent, antidepressant, antidiabetic agent, anti-parasitic, antidiarrheal agent, antimigraine agents, antipsychotic, antiparkinsonian agent, anxiolytic, or hypotensive agent.

In an exemplary embodiment, the drug delivery system comprises an anti-viral agent adsorbed to HPAC. For example, the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine or sofobuvir.

In another embodiment, the provided drug delivery system a live virus, live-attenuated virus or a fixed virion adsorbed to HPAC. For example, the virus, live-attenuated virus or fixed viron is Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus. In some embodiments, the provided drug delivery systems further comprise an adjuvant.

The disclosure provides for methods of treating a viral infection comprising administering highly porous activated carbon in an amount that inhibits virus entry into a cell or reduces viral spread. In any of these methods, the HPAC is administered alone or with an anti-viral agent or other therapeutic agent, such as with a drug delivery system in which an anti-viral agent or therapeutic agent is adsorbed within the HPAC.

The disclosure also provides for methods of treating a condition or disorder comprising the step of administering a therapeutically effective amount of any of the disclosed compositions which comprise HPAC and a therapeutic agent, any of the disclosed drug delivery systems with comprise a therapeutic agent adsorbed to HPAC. For example, the condition is a viral infection.

In any of the disclosed methods, the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine or sofobuvir.

In any of the disclosed methods, the HPAC, composition or drug delivery system is administered by parenteral, nasal, oral, pulmonary, topical, vaginal, or rectal delivery. In some embodiments, the HPAC, composition or drug delivery system is administered by sustained released. In additional embodiments, the HPAC, composition or drug delivery system is administered prophylactically. In other embodiments, HPAC, composition or drug delivery system is administered. In a particular example, the therapeutic administration neutralizes a virus.

The disclosure provides for compositions for treating a viral infection, wherein the composition comprises highly porous activated carbon in an amount that inhibits virus entry into a cell or reduces viral spread. For example, the therapeutically effective amount reduces or prevents virus replications, reduces viral load, reduces or prevents viral spread, e.g. inhibiting syncytia formation, and/or reduces or prevents viral entry into a cell. In any of these compositions, an anti-viral agent or other therapeutic agent is optionally adsorbed within the HPAC.

The disclosure also provides for compositions for treating a condition or disorder comprising a therapeutically effective amount of any of the disclosed compositions which comprise HPAC and a therapeutic agent, any of the disclosed drug delivery systems which comprise a therapeutic agent adsorbed to HPAC. For example, the therapeutically effective amount treats, reduces or prevent the onset, duration or effect of symptoms of the condition or disorder and/or the onset, duration or effect of the disease, condition or disorder itself.

In any of the disclosed compositions, the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine and sofobuvir or combinations thereof.

In any of the disclosed compositions for treating a viral infection or treating a disorder, the HPAC, composition or drug delivery system is formulated for parenteral, nasal, oral, pulmonary, topical, vaginal, or rectal delivery. In some embodiments, the HPAC, composition or drug delivery system is administered by sustained released. In additional embodiments, the HPAC, composition or drug is administered prophylactically or therapeutically. In a particular embodiment, the therapeutic administration neutralizes the virus.

The disclosure provides for use of highly porous activated carbon (HPAC) for the preparation of a medicament for the treatment of a viral infection, wherein the HPAC is in an amount that inhibits virus entry into a cell or reduces viral spread. In some embodiments, the HPAC is in an amount that reduces or prevents virus replication, reduces viral load, reduces or prevents viral spread, e.g. inhibiting syncytia formation, and/or reduces or prevents viral entry into a cell. In any of the disclosed medicaments, an anti-viral agent or other therapeutic agent is optionally adsorbed within the HPAC.

The disclosure also provide for use of a therapeutically effective amount of any of the disclosed compositions which comprise HPAC and a therapeutic agent or any of the disclosed drug delivery systems with comprise a therapeutic agent adsorbed to HPAC, for the preparation of a medicament for the treatment of the condition or disorder. For example, the therapeutically effective amount treats, reduces or prevent the onset, duration or effect of symptoms of the condition or disorder. In a particular embodiment, the condition is a viral infection.

In any of the disclosed medicaments, the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine or sofobuvir.

In any of the methods of treating a viral infection, compositions for treating a viral infection or uses for preparation of a medicament for treating a viral infection, the infection is caused Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

In any of the disclosed the uses, the medicament is formulated for parenteral, nasal, oral, pulmonary, topical, vaginal, or rectal administration. In some embodiments, the HPAC, composition or drug delivery system is administered by sustained released. In additional embodiments, the HPAC, composition or drug is administered prophylactically or therapeutically. In some embodiments, the therapeutic administration neutralizes the virus.

In any of the methods, compositions for treating a viral infection or a disorder or condition, or use for preparation of a medicament for treating a viral infection or a disorder or condition, the HPAC is a microporous carbon. In some embodiments, the HPAC has a pore size ranging from about 10 Å to about 20 Å, or has a cumulative pore volume ranging from about 25 cc/g to about 0.75 cc/g or has a total pore volume ranging from about 500 m²/g to about 3000 m²/g. In addition, the HPAC has a particle size distribution ranges from about 10 nM to about 500 μM.

In an additional embodiment, the disclosure provides for methods of eliciting an immune response comprising administering a vaccine composition to a subject in need, wherein the vaccine composition comprises i) a live virus, a live-attenuated virus or a fixed virion, and ii) highly porous activated carbon, wherein the vaccine elicits an immune response in the subject. In some embodiments, the vaccine composition further comprises an adjuvant. In addition, the vaccine composition is administered by oral, nasal, vaginal, rectal, ocular or sublingual route.

The disclosure also provides for composition for eliciting an immune response comprising i) a live virus, a live-attenuated virus or a fixed virion, and ii) highly porous activated carbon. In some embodiments, the composition further comprises an adjuvant. In addition, the composition is formulated for administration by oral, nasal, vaginal, rectal, ocular or sublingual route.

In another embodiment, the disclosure provides for use of a vaccine composition for the preparation of a medicament for eliciting an immune response in a subject in need, wherein the vaccine composition comprises i) a live virus, a live-attenuated virus or a fixed virion, and ii) highly porous activated carbon, wherein the vaccine elicits an immune response in the subject. In some embodiments, the vaccine composition further comprises an adjuvant. In addition, the is medicament is formulated for adminstration by oral, nasal, vaginal, rectal, ocular or sublingual route.

In any of the methods, compositions for eliciting an immune response or use for preparation of a medicament for eliciting an immune response, the virus, live-attenuated virus or fixed virion is Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

In any of the methods, compositions for eliciting an immune response or use for preparation of a medicament for eliciting an immune response, the HPAC is a microporous carbon. In some embodiments, the HPAC has a pore size ranging from about 10 Å to about 20 Å, or has a cumulative pore volume ranging from about 25 cc/g to about 0.75 cc/g or has a total pore volume ranging from about 500 m²/g to about 3000 m²/g. In addition, the HPAC has a particle size distribution ranges from about 10 nM to about 500 μM.

### BRIEF DESCRIPTION OF THE DRAWINGS

FIGS. 1A-1K provides data demonstrating prophylactic, neutralization and therapeutic efficacy of HPAC. (A) Fluorescence imaging of GFP-HSV-1 and GFP-HSV-2 infected HCE or HeLa cells treated with 1 mg/mL HPAC prophylactically. (B) GFP HSV-1 and HSV-2 viruses were neutralized with 1 mg/mL HPAC prior to its application to HeLa cells and the viral entry was measured. Blue—DAPI, Red—drg staining of Actin, Green—GFP Virus. (C) HCEs and HeLas were infected with HSV-1 and HSV-2 respectively for a period of 2 hours before the addition of Mock PBS or HPAC at 1 mg/mL. 24 hpi, fluorescence images were taken to understand the extent of what iviral spread in HPAC treated samples compared to mock. Green—17 GFP HSV-1 or HSV-2 333 GFP virus. Viral entry for prophylactic (D) and neutralization treatments (E) was quantified using a b-galactosidase reporter virus. ((F) Intracellular viral load for HPAC therapeutic treatent was quantified using a plaque assay. Representative immunoblots of HSV-1 (G) or HSV-2 gB (H) protein and human glyceraldehyde-3-phosphate dehydrogenase (GAPDH) from infected HCE or HeLa cells treated with varying concentrations of HPAC. (I) Toxicity of HPAC in vitro was evaluated MTT assay after the incubation of multiple concentrations of HPAC with HCEs, HeLa, VK2 and HFF cells for 24 hours. (J) CHO cells were categorized into two populations: effector (green) and target (red) cells. Effector cells express HSV-1 glycoproteins (gD, gB, gH, and gL) and T7 polymerase, whereas the target cells express nectin-1 (a gD receptor) and the luciferase gene under T7 promoter. Effector CHO cells were pre-treated with mock PBS or varying amounts of HPAC for 30 min before they were added to the target CHO cells. Images of the syncytial cluster were taken by dyeing the cells with NucBlue live cell nucleus stain. (K) Optical density measurements were performed by serially diluting HPAC in a 96 well plate and measuring the optical absorbance at 650 nm. Lower absorbance corresponds to a clearer solution. Data are represented as mean±SD. Significance to mock-treated cells was determined by one-way ANOVA followed by Dunnett's multiple comparisons test (n=3 replicates)

FIG. 2 demonstrates HPAC strongly binds to HSV-1 GFP virus. Imaged at 100× on a confocal microscope.

FIG. 3 demonstrates extracellular virus-based plaque assay for HPAC therapy. HCEs and HeLa cells were infected with HSV-1 or HSV-2 respectively for 2 hours before difference concentrations of mock PBS or HPAC was therapeutically added. 24 hours post infection, supernatants from the treated samples were collected and overlaid on Vero cells to perform a Plaque assay.

FIG. 4 demonstrates HPAC inhibits HSV induced syncytia. CHO cells transiently transfected with either gB, gD, gH, gL, and T7 polymerase or nectin-1 and T7 promoter. 24 hours post transfection, both the CHO cell types were mixed into a single well in the presence of varying concentrations of HPAC. 24 hours post HPAC addition, the cells were lysed and luciferin was added to analyse the extent of luciferase production. Greater intensity implies higher amount of syncitia.

FIG. 5 demonstrates HPAC at lower concentrations has low optical density. Various stock concentrations of HPAC were prepared in PBS and their optical density was measured using a standard plate reader at 450, 560 and 600 nm.

FIGS. 6A-6B demonstrates HPAC does not elevate interferons in HCEs (A) and HFFs (B) as measured by quantitative RT-PCR. Cells were either incubated with mock PBS (negative control), HPAC or HSV-1 (positive control). 24 hours post incubation, cells were lysed in TRlzol reagent and RNA was isolated using standard instructions.

FIG. 7A-7G demonstrate Acyclovir (ACV) release from DECON is triggered by the addition of virus. (A) provides ACV standard curve generated by UV absorbance at 252 nm. (B) demonstrates ACV release from HPAC was measured by dispersing DECON. (C) Graphical representation of DECON drug release testing. (D) provides representative fluorescence images of HSV-1 infected cells treated with the supernatant (top) or DECON pellet (bottom) on days 2, 4 and 7. Green represents 17GFP HSV-1 virus. Scale bar similar for all the images; 100 bim (E) provides flow cytometry data on HCE cells infected with HSV-1, non-infected (GFP-negative) cells were used as negative control and infected-nontreated (GFP-positive) were used as positive control. The panel on the right side indicated in green color represent the number of cells infected in each treatment group. (F) Representative immunoblots from samples treated with Supernatant or DECON pellet on respective days. (G) Varying concentrations of purified virus and cell debris were added to fresh 1 mg/mL DECON to estimate if they triggered ACV release from DECON. (H) Burst and sustained release profiles for DECON. ACV release was estimated using the supernatants from these samples for a period of 24 hours.

FIG. 8A-8H demonstrate prophylactic or therapeutic use of DECON protects from herpes infections in vitro. FIG. 8A provides a representative image pointing to the optical density and concentration of DECON used for these set of experiments. FIG. 8B provides fluorescent images showing extent of HSV-1 infection (green) in HCEs treated with either ACV loaded DECON, DMSO loaded DECON, HPAC alone, mock DMSO, prophylactically added ACV or therapeutically added ACV. Scale bar is similar for all images; 100 μm. FIG. 8C provides flow cytometry data on the samples at 24 hpi showing the extent of cells infected with HSV-1 GFP. FIG. 8D provides representative immunoblots for samples showing HSV-1 gB protein in comparison with GAPDH for HSV-1 infected HCEs at 24 hpi. FIGS. 8E-8G provide plaque assay data showing extent of virus inhibition by DECON in comparison to ACV, HPAC and mock DMSO for HSV-2, pseudo rabies virus (PRV) and bovine herpes virus (BHV) for intracellular virus collected 24 hpi. Data are represented as mean±SD. Significance to mock-treated cells was determined by one-way ANOVA followed by Dunnett's multiple comparisons test (n=2 replicates). FIG. 8H provides the full length blots for the Western blot shown in FIG. 8D.

FIG. 9 demonstrates DECON was effective when added 24 hours post infection.

FIG. 10A-10I demonstrate alternate day ocular dosing with DECON curbs HSV-1 in a murine model of ocular infection. FIG. 10A provides images of the ocular region taken on days 0, 7 and 14 for mice infected and treated as stated above. FIG. 10B provides a representative image of the DECON concentration used for this set of experiments (top) is shown. FIG. 10C provides ocular washes, collected on days 2, 4 and 7, analyzed for the presence of virus through plaque assays. FIG. 10D provides a Kaplan-Meier survival curves for the infected and treated mice. FIG. 10E provides a disease scores (0-4; 4 being severe) taken on days 2, 4, 7, 10, 14 and 21 were scored in a blinded fashion. (F) Mice were sacrificed on day 21 and their eyes were frozen in OCT medium for histology. 10 micron sections of the eye for all the groups were taken and stained with hematoxylin and eosin stain. FIGS. 10G and 10H provide Draining lymph nodes isolated from mice either mock infected or HSV-1 infected and either mock treated or DECON treated were photographed and weighed. FIG. 10I shows corneal sensitivity measured (n=5 per treatment group). Lower scores indicate loss in corneal sensitivity.

FIG. 11 demonstrates DECON protects the murine cornea from HSV-1 infection. 10 μM ocular sections were stained with H&E stain. Top images were taken at 2.5× magnification and bottom bars are 20× magnifications of the same image.

FIG. 12A-12F demonstrates topical vaginal administration of DECON on alternate days was as effective as daily systemic ACV dosing. FIG. 12A provides representative stereoscope images of the murine genital region 0 and 7 days post infection. FIG. 12B Vaginal swabs collected on days 2, 4 and 7 were overlaid on Vero cells to estimate extent of virus production through plaque assays. (C) Disease scores were given in a blinded fashion from 0 to 4; 4 being severe. (D) Kaplan-Meier survival curves for the infected and treated mice. (E) Draining lymph nodes for the infected and treated mice were collected on day 21, washed, photographed and weighed (F).

FIG. 13 provides a graphical abstract showing DECON protecting cells against viral infection. When cells are treated with DECON particles during HSV infection, DECON binds to the surface of the cells and is protected from rapid clearance in the corneal and vaginal epithelium. While on the surface, DECON traps incoming viruses. The act of trapping the virus ensues the release of ACV in to the surrounding cells resulting in their protection from viral infection. While in non-treated or topically treated (with non-DECON drugs) cells, there is no deterrent to the incoming virus which results in infection, replication and subsequent inflammation of the corneal or vaginal epithelium. Given that HPAC and DECON do not entail an immune response by themselves; they will be safe to use in ocular drops or genital ointments.

FIG. 14 demonstrates DECON loaded with nucleoside analogs was overlaid on HSV-1 GFP infected cells. The cells were collected and analyzed using flow cytometry for the extent of GFP fluorescing (infected) cells. The red-line demarcates the non-infected from the infected population.

### DETAILED DESCRIPTION DISCLOSURE

Activated charcoal is highly porous in nature and has a surface area far greater than any other nanoparticle or microparticle known to material science. Furthermore, carbonization at very high temperatures in the presence of steam activates every pore present on the surface of charcoal. On an average, iodine absorption coefficient of activated charcoal is greater than 1, suggesting it can absorb more iodine (or other effluent molecules) than its own weight. Given this property, activated charcoal has been widely used in various industries including clinics and emergency medical departments. Clinically, it is used as emergency medication to treat drug overdose since it adsorbs specific drugs in large amounts thereby preventing their absorption in the human gut. Their role as scavenging systems for poison ingestion has proven to be a lifesaving emergency treatment. Activated charcoal is also currently available as a prescription-free dietary supplement for relieving gastric trouble and bloating of the stomach, although their efficacy has not been scientifically tested. Furthermore, owing to their toxin scavenging properties, their use as gut cleansers in health drink industry has also seen upsurge.

Few studies in the past have reported the use of activated charcoal in the treatment of sexually transmitted diseases (STDs). U.S. Patent Application Publication No. US 20030003095 describes methods of preventing pregnancy and preventing a sexually transmitted disease (STD) by administering safe and effective amount of activated charcoal to the subject in need. While the methods in this application contemplate use of activated carbon to bind many types of STD pathogens, the examples focus on preventing bacterial infections such as C. trachomatis and N. gonorrhoeae. In another study by Kunihiko et al, an unblinded prospective randomized controlled 10-day trial was designed to study 64 women with bacterial vaginosis (Tominaga et al. Pers, Med. Univ. 1:54-57, 2012). The study was divided in to two groups where one group was treated with 100 mg Chloramphenicol, and the other group was treated with trans-vaginal tampons dipped in 10% w/v activated charcoal (AC) solution. While both treatments successfully reduced the amount of vaginal secretion and showed significant improvements in the magnitude of malodor, 27/32 (84%) women from the group treated with chloramphenicol completely lost the vaginal flora (lactobacilli). However only 3.1% of those treated with AC lost vaginal flora indicating a potentially promising treatment for this infection without adverse effects.

The studies provided herein demonstrate that highly porous activated carbon (HPAC) particles restrict HSV-1 and HSV-2 from entering target cells and reduced virus cell spread by inhibiting syncytia formation. Charcoal black is a common ingredient used in the cosmetic industry, especially for the eye. Traditionally, black soot obtained from burning clarified butter was used as an eye-liner and is still used today in various cultures across the world. There is a need to identify enhanced delivery methods for antiviral drugs; and the present disclosure utilizes charcoal in its surface-active form to trap virions and provide antiviral effects.

### Highly Porous Activated Carbon

Activated charcoal is highly porous in nature and has a surface area far greater than any other nanoparticle or microparticle known to material science. Furthermore, carbonization at very high temperatures in the presence of steam activates every pore present on the surface of charcoal. On an average, iodine absorption coefficient of activated charcoal is greater than 1, suggesting it can absorb more iodine (or other effluent molecules) than its own weight.

The term “highly porous activated carbon (HPAC)” refers to a carbonaceous, highly porous adsorptive medium. The networks of pores in HPAC are channels created within a rigid skeleton of disordered layers of carbon atoms, linked together by chemical bonds, stacked unevenly, creating a highly porous structure of nooks, crannies, cracks and crevices between the carbon layers.

HPAC is generally obtained from a carbonaceous source material such as bamboo, coconut husk, willow, peat, soft and hard wood, coir, lignite coal, bituminous coal, olive pits and petroleum pitch. It is generated by physical activation using heated gas, such as using carbonization wherein the carbon content is pyrolyzed at temperatures ranging 600-900° C. or using activation/oxygenization wherein the carbon content is exposed to oxidizing atmosphere at temperatures about 250° C. (usually ranging 600-1200° C.). HPAC is also generated by chemical activation, wherein the carbon source is impregnated with a chemical such as acid, strong base or a salt, e.g. phosphoric acid, potassium hydroxide, sodium hydroxide, calcium chloride or zinc chloride, and then exposed to temperatures ranging 250-600° C.

The HPAC is provided in microparticlate form or nanoparticlate form. For example, the microparticulate form is particles greater than 1000 microns In addition, the HPAC is granular or a powder.

The HPAC may be nanoporous with a pore width of less than 1 nm, microporous with a pore width of less than 2 nm, mesoporous with a pore width ranging from about 2 to about 50 nm, or macroporous with a pore width ranging from about 50 nm to about 1 μm. For example, the HPAC has micropores that have a radius less than 1 nm, or mesopores that have a radius that ranges from about 1 nm to about 25 nm or macropores that are greater than 25 nm. In some embodiments, the HPAC has a pore size indicating a microporous carbon with a peak ranging from about 10 Å to about 20 Å, or about 12 Å to about 18 Å, or about 13 Å to about 15 Å, or about 14 Å to about 16 Å, or about 10 Å to about 15 Å, or about 11 Å to about 16 Å, or about 12 Å to about 17 Å, or about 13 Å to about 18 Å, or about 14 Å to about 19 Å. In addition, the HPAC has a pore size indicating a microporous carbon with a peak of about 10 Å, or about 11 Å, or about 12 Å, or about 13 Å, or about 14 Å or about 15 Å, or about 16 Å, or about 17 Å, or about 18 Å, or about 19 Å, or about 20 Å.

In addition, the HPAC ha a low total pore volume ranging from 500 m²/g to about 1000 m²/g, or from 550 m²/g to about 950 m²/g, or from 600 m²/g to about 800 m²/g, or from about 560 m²/g to about 860, or from 670 m²/g to about 870 m²/g or from about 700 m²/g to about 1000 m²/g, or about 800 to about 1000 m²/g, or about 900 m²/g to about 1200 m²/g, or about 1000 m²/g to about 1500 m²/g, or about 1200 m²/g to about 1800 m²/g, or about 1500 to about 2000 m²/g, or about 2000 m²/g to about 3000 m²/g, or about 1000 m²/g to about 3000 m²/g. The low total pore volume is about 500 m²/g, about 550 m²/g, about 600 m²/g, about 650 m²/g, about 700 m²/g, about 750 m²/g, about 800 m²/g, about 825 m²/g, about 850 m²/g, about 860 m²/g, about 875 m²/g, about 900 m²/g, about 950 m²/g, about 1000 m²/g, about 1200 m²/g, about 1500 m²/g, about 1800 m²/g, about 2000 m²/g, about 2200 m²/g, about 2500 m²/g, 2800 m²/g or about 3000 m²/g.

In some embodiments, the cumulative pore volume is about 0.25 cc/g to about 0.75 cc/g, or about 0.3 cc/g to about 0.7 cc/g, or about 0.325 cc/g to about 0.675 cc/g or about 0.35 cc/g to about 0.65 cc/g, or about 0.375 cc/g to about 0.625 cc/g, or about 0.4 cc/g to about 0.6 cc/g, or about 0.425 to about 0.625, or about 0.45 cc/g to about 6.5 cc/g, or about 0.475 cc/g to about 0.7 cc/g, or about 0.5 cc/g to about 0.8 cc/g. The cumulative pore volume is about 0.25 cc/g, or about 0.275 cc/g, or about 0.3 cc/g, or about 0.325 cc/g, or about 0.35 cc/g, or about 0.375 cc/g, or about 0.4 cc/g, or about 0.425 cc/g, or about 0.45 cc/g, or about 0.475 cc/g, or about 0.5 cc/g, or about 0.525 cc/g, or about 0.55 cc/g, or about 0.575 cc/g, or about 0.6 cc/g, or about 0.625 cc/g, about 0.65 cc/g, or about 0.675 cc/g, or about 0.7 cc/g, or about 0.725 cc/g, or about 0.75 cc/g, or about 0.8 cc/g.

For example, the disclosure provides for HPAC having a pore size indicating a. microporous carbon with peak about 13 Å and low total pore volume of 861 m²/g. The cumulative pore volume was 0.45 cc/g which is approximately 30% of other high efficiency carbons.

In some embodiments, the HPAC is sterilized for medical administration. For example, the HPAC is gamma irradiated (technique to sterilize carbon used for medical applications). Alternatively, the HPAC is sterilized by steam, heat, peroxide, or gases such as ethylene oxide, ozone, mixed oxides of nitrogen or chlorine dioxide.

In some embodiments, the HPAC is basic, such as having a pH ranging from about 8 to about 9, or from about 8.5 to about 10, or from about 9 to about 11. For example, the pH of HPAC is about 8.0, or about 8.1, or about 8.2, or about 8.3, or about 8.4 or about 8.5 or about 8.6, or about 8.7, or about 8.8 or about 8.9 or about 9.0.

In some embodiments, gravimetric analysis (TGA) indicates HPAC is about 1% ash to about 25% ash, about 5% ash to about 20% ash, about 1% ash to about 10% ash, about 5% ash to about 15% ash, about 10% to about 20% ash, about 15% to about 35% ash, or about 15% to about 25% ash, or about 15% to about 20% ash, or about 20% to 35% ash, or about 25% to 35% ash. For example, the HPAV is about 1% ash, about 2% ash, about 3% ash, about 4 ash, about 5% ash, about 6% ash, about 7% ash, about 8% ash, about 9% ash, about 10% ash, about 11% ash, about 12% ash, about 13% ash, about 14% ash, about 15% ash, about 16% ash, about 17% ash, about 18% ash, about 19% ash, about 20% ash, about 21% ash, about 22% ash, about 23% ash, about 24% ash, about 25% ash, about 26% ash, about 27% ash, about 28% ash, about 29% ash, about 30% ash, about 31% ash, about 32% ash, about 33% ash or about 35% ash.

Particle size distribution refers an index (means of expression) indicating the size of the particles present and the proportion of sizes present in a sample of granular material or of powder. Volume, area, length and quantity may be used as standards for determining particle size distribution. In some embodiments, the HPAC has an observed d₅₀ of 4.6 μm which indicates that the carbon was not custom ground.

The HPAC is provided in a variety of shapes and sizes. For example HPAC is provided as granules, fibers, and beads. Furthermore, HPAC is provided in the shape of a sphere, polyhedron, cylinder, as well as other symmetrical, asymmetrical, and irregular shapes. In addition, the HPAC is provided in more complex forms such as webs, screens, meshes, non-wovens, wovens, and bonded blocks.

As provided herein, HPAC has antiviral activity and is used as a drug delivery system. HPAC is generally regarded as safe for human use by the FDA and adverse effects due to their exposure have seldom been reported. The toxicological concentration 50 (TC50) value is considered well above 1 g/kg body weight for adults and 0.5 g/kg body weight for infants. This accounts for a TC50 value greater than 50 mg/mL of HPAC for human use. Pro-kidney health benefits also make HPAC an attractive platform for drug delivery especially in cases where renal failure has been reported after the prolonged use, such as for a drug such as ACV.

The studies described herein investigated whether HPAC could act as a prophylactic and/or therapeutic treatment against both HSV-1 and HSV-2 infection. It was demonstrated that HPAC was able to efficiently restrict viral entry into cells when added prophylactically (added prior to infection) to the cell surface. When added therapeutically (added post infection), HPAC showed potent inhibition in viral spread and replication. Furthermore, the efficiency of HPAC in limiting cell to cell syncytial fusion using a virus free transfection assay and trapping the virus using a neutralization entry assay. Results from these studies showed that HPAC was not only able to restrict syncytia formation but also trapped virus efficiently in its nanopores.

### Drug Loading of HPAC

Another aspect of the disclosure provides for compositions comprising HPAC and one or more therapeutic agents. In addition, the disclosure provides for drug delivery systems comprising one or more therapeutic agents and HPAC. In an exemplary embodiments, one or more therapeutic agents are adsorbed within HPAC.

The term “adsorption” refers to the attachment or adhesion of the therapeutic agent to the surface of the HPAC. The porosity of activated carbons offers a vast surface on which this adsorption can take place. Adsorption occurs within the pores of the HPAC, and the pores should be slightly larger than the therapeutic agent. The therapeutic agent is adsorbed, encapsulated or trapped within the internal pore structure of HPAC by Van Der Waals Forces or other bonds of attraction and accumulate onto a solid surface of HPAC.

One advantage of using HPAC as a drug delivery system is that the extensive pore system provides a vast surface area for adsorption of therapeutic agents. Another advantage using HPAV as a drug delivery system is that it allows for sustained release of the therapeutic agent upon contact with a living organism.

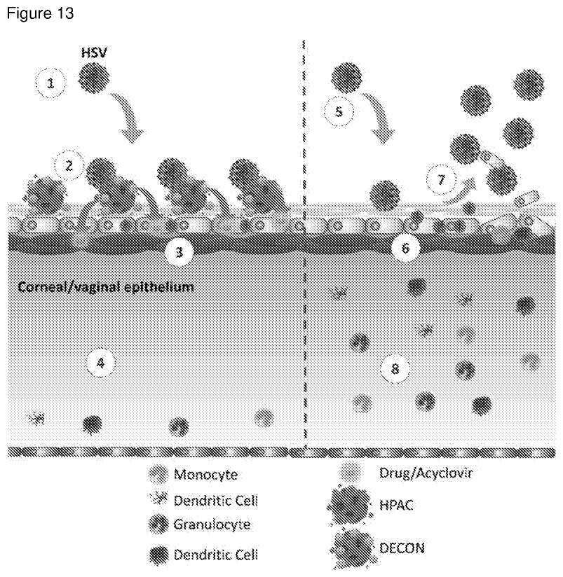

In an exemplary embodiment, the anti-viral agent was loaded on HPAC. To test synergistic benefits as well as drug loading potential of HPAC, acyclovir (ACV) was incubated with HPAC (DECON). Quite interestingly, HPAC was able to efficiently adsorb ˜100% of the drug leaving no traces of ACV in free solution. HPAC showed slight signs of drug release from its nanopores over an incubation period of 7 days. While the drug loading efficiency was promising for HPAC, drug release studies initially damped enthusiasm regarding its applicability as a drug delivery agent. DECON particles were tested for antiviral activity and to our surprise saw that DECON exhibited antiviral efficacy whether added prophylactically or therapeutically to HSV infected cells (both HSV-1 and HSV-2). In contrast, prophylactically added ACV did not elicit a robust antiviral response, unlike that observed when added therapeutically. The robust antiviral activity seen from DECON may be a result of its charged porous surface interacting with the cell's surface, inducing an active exchange of ions (Na⁺, K⁺, Ca⁺, Cl⁻, OH⁻) and resulting in sustained or slow release of ACV from its pores. Alternatively, given that HPAC efficiently traps HSV, we showed that the addition of virus to DECON treated cells triggers ACV release from its pores when virus is captured in them (FIG. 13).

These results by DECON prompted us to perform antiviral efficacy studies in vivo in murine model of ocular (HSV-1) and genital (HSV-2) infection. Typical anti-HSV studies administer compounds 2 to 6 times a day to achieve effective response. However, to show that DECON particles can effectively stick and deliver drugs over a prolonged period of time, we administered DECON on alternate days topically. As expected, we saw significant differences with reduced infection upon DECON treatment. For ocular model of HSV-1 infection, groups treated with HPAC and TFT on alternate days did not show any significant reduction in viral replication when compared to mock treated groups. Furthermore, DECON treatment during vaginal HSV-2 infection showed equal or better therapeutic efficacy than systemically administered ACV. It is also important to note that while topical administration of DECON was effective when administered on alternate days, ACV treatment was effective only when administered every day for a period of 7 days. In conclusion, our drug delivery model (FIG. 13) hypothesizes that when HSV (FIG. 13—step 1/5) infects corneal/vaginal epithelium, the presence of DECON (FIG. 13—step 2) helps capture the virus and releases the encapsulated drug (FIG. 13—step 3) providing a dual protection (FIG. 13—step 4) to the treated cells. While non-treated tissue gets infected (FIG. 13—step 6), leading to viral shedding (FIG. 13—step 7) which in-turn causes inflammation and immune cell infiltration in to the infected tissue (FIG. 13—step 8).

### Sustained Release

The composition comprising HPAC and the drug delivery systems disclosed herein release a therapeutic agent by immediate release, controlled release, sustained release, extended release, delayed release, or bi-phasic release formulation. Methods of formulating compounds for controlled release are known in the art. See, for example, Qian et al., J Pharm 374: 46-52 (2009) and International Patent Application Publication Nos. WO 2008/130158, WO2004/033036; WO2000/032218; and WO 1999/040942.

As used herein, the term “sustained release” is characterized by the gradual release of the therapeutic agent from the HPAC particles of the composition over an extended period of time, optionally greater than about 30 minutes. With sustained release, the rate of release of the therapeutic agents from the HPAC particles is controlled in order to maintain activity of the therapeutic agents for a longer period of time. In some embodiments, the HPAC may release greater than about 40% of the one or more therapeutic agents over a period of about 6 hours or more.

For example, sustained release compositions allow delivery of a therapeutic agent to a subject over an extended period of time. Such release rates can provide therapeutically effective levels of a therapeutic agent for an extended period of time and thereby provide a longer period of pharmacologic or diagnostic response as compared with conventional rapid release dosage forms. Such longer periods of response provide for many inherent benefits that are not achieved with immediate release dosages. In using analgesics for treatments of chronic pain, controlled release formulations can be preferred over conventional short-acting formulations.

For sustained release, the HPAC compositions may be compressed into tablets (regular tablets, oral-disintegrating tablets (ODT), self-disintegrated tablets, chewable tablets), filled into capsules (conventional hard gelatin capsules and easy open capsules to sprinkle) or loaded into stick packs to sprinkle over food or into water or other liquid drinks. In some embodiments, the one or more intra-granular excipients may comprise one or more diluents, binders, fillers, surfactants/emulsifying agents, disintegrating agents, or a combination thereof. In certain embodiments, the one or more intra-granular excipients may comprise one or more cellulose-derivative diluents. Examples of cellulose-derivative diluents may include lactose, isomalt, cellulose, starch, cyclodextrin, mannitol, and sorbitol.

### Vaccine Compositions

The experiments described herein demonstrate that HPAC efficiently entraps virus and therefor the disclosure provides for a method of delivering virus-based vaccines, and in particular mucosal vaccine. For example, the disclosure provides for vaccine compositions for delivering a virus-based vaccine to a subject in need thereof, wherein administration of the vaccine composition elicits an immune response in the subject. The vaccine composition comprises a live virus, live-attenuated virus, or fixed virions adsorbed within HPAC, and optionally comprises an adjuvant.

The term live viruses refer to a virus that can infect, replicate and propagate in a host. Attenuated viruses are viruses that have been modified either physically, such as by heat or UV treatment, or chemically modification such as treating with a chemical to destroy its proteins. These modifications ensure that the attenuated virus no longer infects, replicates or propagates due to the destruction of its cellular or genetic structure.

The term live-attenuated virus refers to those viruses that have either been physically trapped in a structure making them unable to infect a host or those viruses that have been genetically modified in such a way that some or most of their proteins and/or nucleic acids are absent or non-functional. Due to this entrapment or genetic modification, the viruses are unable to either infect, replicate or propagate inside the host.

The term fixed virion refers to a virus particle refers to the process of disabling any physical movement of the virus from a given structure. Once a virion is fixed, it cannot detach itself from the structure that it has been fixed to. A solitary fixed virion is referred to a virion that has been chemically treated in such a way that although it is not attached to any structure, none of its proteins and/or cellular components are fixed in place and do not move and hence cannot infect, replicate or propagate inside a host.

Known adjuvants include, for example, emulsions such as Freund's Adjuvants and other oil emulsions, Bordetella pertussis, MF59, purified saponin from Quillaja saponaria (QS21), aluminum salts such as hydroxide, phosphate and alum, calcium phosphate, (and other metal salts), gels such as aluminum hydroxide salts, mycobacterial products including muramyl dipeptides, solid materials, particles such as liposomes and virosomes. Examples of natural and bacterial products known to be used as adjuvants include monophosphoryl lipid A (MPL), RC-529 (synthetic MPL-like acylated monosaccharide), OM-174 which is a lipid A derivative from E. coli, holotoxins such as cholera toxin (CT) or one of its derivatives, pertussis toxin (PT) and heat-labile toxin (LT) of E. coli or one of its derivatives, and CpG oligonucleotides. Adjuvant activity can be affected by a number of factors, such as carrier effect, depot formation, altered lymphocyte recirculation, stimulation of T-lymphocytes, direct stimulation of B-lymphocytes and stimulation of macrophages.

The disclosed vaccine are mucosal vaccine administered by oral, nasal, vaginal, rectal, ocular and sublingual routes.

In other embodiments, the vaccine compositions are typically formulated as injectables, either as liquid solutions or suspensions; solid forms suitable for solution in, or suspension in, liquid prior to injection may also be prepared. The preparation may also be emulsified. The active immunogenic ingredient is often mixed with excipients, which are pharmaceutically acceptable and compatible with the active ingredient. Suitable excipients are, e.g., water, saline, dextrose, glycerol, ethanol, or the like and combinations thereof. In addition, if desired, the vaccine may contain minor amounts of auxiliary substances such as wetting or emulsifying agents, pH buffering agents, or adjuvants, which enhance the effectiveness of the vaccine. The vaccines are conventionally administered parenterally, by injection, for example, either subcutaneously or intramuscularly.

The live and live-attenuated virus include Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, hepatitis D, hepatitis E, and human immunodeficiency virus. Viruses also include additional herpesviruses: Cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43. Additional virus include Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria whaturgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses.

Bovine and Porcine virus include Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV, Venezuelan equine encephalomyelitis VEE.

### Pharmaceutical Formulations and Modes of Administration

As used herein, “subject” refers to any mammal, including humans, domestic and farm animals, and zoo, sports, or pet animals, such as dogs, horses, cats, sheep, pigs, cows, etc. The preferred mammal herein is a human, including adults, children, and the elderly. Preferred sports animals are horses and dogs. Preferred farm animals are cows, pigs, horses, goats and sheep. Preferred pet animals are dogs and cats.

As used herein, a “therapeutically effective amount” in reference to the disclosed HPAC compositions or therapeutic agents refers to the amount sufficient to induce a desired biological, pharmaceutical, or therapeutic result. That result can be treating, reducing or preventing of the signs, symptoms, or causes of a disease or disorder or condition, or any other desired alteration of a biological system. In regards to anti-viral agents, a therapeutically effective amount reduces or prevents virus replications, reduces viral load, reduces or prevents viral spread, e.g. inhibiting syncytia formation, and/or reduces or prevents viral entry into a cell.

As used herein, the terms “treating” and “treatment” refer to both therapeutic treatment and prophylactic or preventative measures.

In exemplary aspects, the HPAC and/or the drug delivery systems disclosed herein is part of a pharmaceutical composition comprising HPAC and a pharmaceutically acceptable carrier, diluent, or excipient. In exemplary aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopedia for use in animals, including humans.

The pharmaceutical composition in various aspects comprises any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In exemplary aspects, the pharmaceutical composition comprises formulation materials that are nontoxic to recipients at the dosages and concentrations employed. In specific embodiments, pharmaceutical compositions comprising HPAC and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition comprises formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as bcnzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbatc, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18” Edition, (A. R. Genrmo, ed.), 1990, Mack Publishing Company.

The pharmaceutical compositions in various instances are formulated to achieve a physiologically compatible pH. In exemplary embodiments, the pH of the pharmaceutical composition is for example between about 4 or about 5 and about 8.0 or about 4.5 and about 7.5 or about 5.0 to about 7.5. In exemplary embodiments, the pH of the pharmaceutical composition is between 5.5 and 7.5.

The pharmaceutical composition may be administered to a subject via parenteral, nasal, oral, pulmonary, topical, vaginal, or rectal administration. The following discussion on routes of administration is merely provided to illustrate exemplary embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. HPAC in various instances is administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight HPAC in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Formulations suitable for oral administration in some aspects comprise (a) liquid solutions, such as an effective amount of HPAC dissolved in diluents, such as water, saline, or orange juice; (b) capsules, sachets, tablets, lozenges, and troches, each containing a predetermined amount of HPAC, as solids or granules; (c) powders; (d) suspensions in an appropriate liquid; and (e) suitable emulsions. Liquid formulations in some aspects include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise HPAC in a flavor, usually sucrose and acacia or tragacanth, as well as pastilles comprising HPAC in an inert base, such as gelatin and glycerin, or sucrose and acacia, emulsions, gels, and the like containing, in addition to, such excipients as are known in the art.

### Topical Delivery

In some embodiments, a composition is formulated, for example, as a topical (e.g., dermal) formulation. In some embodiments, a composition is formulated, for example, for topical administration to a mammal. A topical formulation may include, for example, a formulation such as a gel formulation, a cream formulation, a lotion formulation, a paste formulation, an ointment formulation, an oil formulation, and a foam formulation. The composition further may include, for example, an absorption emollient.

Additional examples of a composition can optionally be formulated to be delivered to the mucosum, or by inhalation, respiration, intranasal, oral, buccal, or sublingual.

Salts may be added. Non-limiting examples of salts include acetate, benzoate, besylate, bitartate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulphate, mucate, napsylate, nitrate, pamoate (embonate, phosphate, diphosphate, salicylate and disalicylate, stearate, succinate, sulphate, tartrate, tosylate, triethiodide, valerate, aluminium, benzathine, calcium, ethylene diamine, lysine, magnesium, megluminie, potassium, procaine, sodium, tromethyamine or zinc.

Topical (skin, e.g., face) formulations can include, for example, a liquid or cream with or without moisturizer. Components of a liquid or cream with moisturizer (moisturizing formulation) can be: Colloidal oatmeal, niacinamide, creamides, phsospholipids, triglycerides, fats or fatty acids, free fatty alcohols, waxes (esters, diesters, triesters, etc.), hydroxyacid diesters, squalene, sterol esters, cholesterol, lactones, etc. In addition, the topical formulations can be incorporated as creams, gels, or foams to serve as topical treatment for viral infection or for rectal or vaginal application (e.g. to mucosal surfaces)

Suitable carrier materials for use in sustained release delivery devices of the disclosure include any carrier or vehicle commonly used as a base for creams, lotions, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

Suitable carriers include: pluronic gels, polaxamer gels, hydrogels containing cellulose derivatives, including hydroxyethyl cellulose, hydroxymethyl cellulose, carboxymethyl cellulose, hydroxypropylmethyl cellulose and mixtures thereof, and hydrogels containing polyacrylic acid (Carbopols). Suitable carriers also include creams/ointments used for topical pharmaceutical preparations, e.g., creams based on cetomacrogol emulsifying ointment. The above carriers may include or exclude, for example, alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

### Therapeutic Agents

The disclosure provides for compositions comprising HPAC and a therapeutic agent. In addition, the disclosure provides for drug delivery systems comprising a therapeutic agent adsorbed within the HPAC. The term “therapeutic agent” refers to any agent or compound known in the art that elicits a therapeutic effect or treats or reduces or prevents the onset, duration or effect of a symptom of a disease, condition or disorder and/or the onset, duration or effect of the disease, condition or disorder itself.

The therapeutic agents include antiviral agents, antibacterial agents, anticancer and cytotoxic agents, analgesics, anti-hypertension drugs, anti-allergenic (anti-histamine), an anti-seizure compound, non-steroidal anti-inflammatory drugs, an antibiotic, growth hormone, parathyroid hormone, insulin, interferons, chemotherapeutic agents, glucagon like peptides (e.g., GLP-1, exenatide), polynucleotides including DNA and RNA such as siRNA and shRNA, plasmids and vectors, DNA based compounds that can target viral/non-viral targets, peptides based on compounds that can target viral/non-viral targets, parathyroid hormones, growth hormones (e.g., IFG and other growth factors), immune suppression agents, and anti-parasitic agents such as various anti-malarial agents. In addition, therapeutic agents include natural enzymes, proteins derived from natural sources, recombinant proteins, natural peptides, synthetic peptides, cyclic peptides, antibodies, receptor agonists, cytotoxic agents, immunoglobins, beta-adrenergic blocking agents, calcium channel blockers, coronary vasodilators, cardiac glycosides, antiarrhythmics, cardiac sympathomemetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thizaolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT₄ partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A₂ inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptase inhibitors, protease inhibitors, anemia, Kaposi's sarcoma, aminoglycosides, carbapenems, cephalosporins, glycopeptides, lincosamides, macrolies, oxazolidinones, penicillins, streptogramins, sulfonamides, trimethoprim and derivatives, tetracyclines, anthelmintics, amebicies, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, antracyclines, actinomycinds, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamine reuptake inhibitors, selective serotonin reuptake inhibitors, antidepressants, Benzisoxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g. lidocaine, articaine hydrochloride, bupivacaine hydrochloride), antipyretics, hynotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opoid antagonists, cholinesterase reactivators, nicotine replacement therapy, antitussives, antiulcer agents and acid suppressants, gastrointestinal drugs, vitamin A analogs and antagonists, vitamin B analogs and antagonists, vitamin C analogs and antagonists, vitamin D analogs and antagonists, vitamin E analogs and antagonists, vitamin K analogs and antagonists or combinations thereof.

The therapeutic agent is an analgesic or a pain relieving agent. Analgesics include ibuprofen, diphenhydramine, acetaminophen, magnesium sailicylate, aspirin, meprobamate, ziconotide, butalbitalor, codeine, hydrocodone, dihydrocodone, oxycodone, naloxone, pentazocine, fentanyl, morphine, hydromorphone, buprenorphine, methadone, meperidine, buprenorphine, oxymorphone, tramadol, nalbuphine, propoxyphene, tapentadol, alfentanil, sufentanil, remifentanil and combinations thereof. Analgesics also include antimigraine agents such as sumatriptan, ergotamine, methysergide maleate, frovatriptan, almoatriptan, ergotamine, rizatraiptan, naproxen, napatripan, eletriptan, zolmitriptan, lasmiditan, dichloralphenazone, isometreptene mucate or combinations thereof. Analgesics also include CGRP inhibitors such as erenunumab (Aimovig), fremanezumab (Ajovy), galcanezumab (Emgality) and epitinezumab. Analgesics include Cox-2 inhibitors such as celecoxib, valdecoxib, and rofecoxib. In addition, analgesics include nonsteroidal anti-inflammatory drugs such as ibuprofen, naproxen, ketoprofen, tometin, etodolac, flurbiprofen, diclofenac, misoprostol, piroxicam, fenoprofen, indomethacin, sulndac, nabumetone, ketorolac, famotidine, mefenamic acid, diflunisal, meloxicam. Analgesics include salicylates such as aspirin, salsalate, magnesium salicylate, choline salicylate, difunisal, and tricosal or combinations thereof.

The therapeutic agent is an anti-allergenic or an anti-histamine, such as cetirizine, desloratadine, ebastine/carebastine, fexofenadine, lovocetirizine, loratadine, mizolastine, rupatadine, Bromp, heniramine, chlorpheniramine, clemastine, diphenhydramine, Ketotifen, naphazoline, pheniramine, azelastine, azelastine, pseudoephedrine, epastine and olopatadine, antihistamine drugs, such as acrivastine, astemizole, cinnarizine, cyproheptadine, dimenhydrinate, flunarizine, meclozine, oxatomide, terfenadine, and triprolidine or combinations thereof.

The therapeutic agent is an antidepressant, such as amoxapine, ciclazindol, maprotiline, mianserin, nortriptyline, trazodone, trimipramine maleate, acetohexamide, chlorpropamide, glibenclamide, gliclazide, glipizide, tolazamide, and tolbutamide or combinations thereof.

The therapeutic agent is an antidiabetic agent, such as acarbose, miglitol, pramlintide, alogliptan, linagliptan, saxagliptin, sitagliptin, albiglutide, dulaglutide, exenatide, liraglutide, lixisenatide, insulin, nateglinide, repaglinide, metformin, canagliflozin, dapagliflozin, empagliflozin, chlorpropamide, glimepiride, glipizide, glyburide, tolazamide, tolbutamide, rosiglitazone, and pioglitazone; alpha-glucosidase inhibitors, biguanides, D-phenylalanine derivatives, dipeptidyl peptidase-4 (DPP-4) inhibitor, Januvia (sitagliptin phosphate), Meglitinide, Sulfonylureas, Diabinese (chlorpropamide), Glucotrol (glipizide), Glucotrol XL (glipizide long-acting), glynase (glyburide), Micronase (glyburide), Thiazolidinediones Actos (pioglitazone) and Avandia (rosiglitazone), combo pills such as Actoplus Met, Avandamet, Avandaryl, Duetact, Glucovance, Janumet, and Metaglip, SGLT-2 inhibitors, Amylin mimetic Symlin (pramlintide acetate), incretin Byetta (exenatide), GLP-1 receptor agonist, Glyxambi, Segluromet, Steglatro, Steglujan, Synjardy, Xigduo XR, Adlyxin, Basaglar, Ozempic, Ryzodeg, Soliqua, Toujeo, Tresiba, and Xultophy or combinations thereof.

The therapeutic agent is an antidiarrheal agent, such as attapulgite, bismuth subgallate, bismuth subsalicylate, loperamide, and diphenoxylate; antidotes; antiemetics, such as hyoscyamine, methscopolamine, scopolamine, cyclizine, dimenhydrinate, hydroxyzine, meclizine, promethazine, dronabinol, nabilone, tetrahydrocannabinol, chlorpromazine, prochlorperazine, alosetron, dolasetron, granisetron, ondansetron, palonosetron, aprepitant, fosaprepitant, rolapitant, dexamethasone, metoclopramide, and trimethobenzamide; antigout agents, such as probenecid, sulfinpyrazone, allopurinol, and colchicine or combinations thereof.

Therapeutic agents include antimigraine agents such as dihydroergotamine mesylate, ergotamine tartrate, methysergide maleate, pizotifen maleate, and sumatriptan succinate; antineoplastic agents, including alkylating agents, antimetabolites, mitotic inhibitors, and hormonal agents, or combinations thereof.

Therapeutic agents include antiparkinsonian agents, such as bromocriptine mesylate and lysuride maleate. The therapeutic agent is an antipsychotics, such as chlorpromazine, fluphenazine, perphenazine, prochlorperazine, thioridazine, trifluoperazine, haloperidol, lithium, loxapine, molindone, pimozide, aripiprazole, asenapine, brexpiprazole, cariprazine, clozapine, iloperidone, lurasidone, olanzapine, paliperidone, pimavanserin, quetiapine, risperidone, and ziprasidone. In addition, therapeutic agents include atypical antipsychotics and corticosteroids, such as beclomethasone, betamethasone, budesonide, cortisone acetate, desoxymethasone, dexamethasone, fludrocortisone acetate, flunisolide, flucortolone, fluticasone, propionate, hydrocortisone, methylprednisolone, prednisolone, prednisone, and triamcinolone or combinations thereof.

The therapeutic agent is an anxiolytic agent such as sedatives and/or hypnotics, such as alprazolam, amyiobarbitone, barbitone, bentazeparn, bromazepam, bromperidol, brotizoiam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenuiixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, suipiride, temazepam, thioridazine, triazolam, and zopiclone or combination thereof.

Therapeutic agents also include hypotensive agents, such as amlodipine, carvedilol, benidipine, darodipine, diltiazem, diazoxide, felodipine, guanabenz acetate, indoramin, isradipine, minoxidil, nicardipine, nifedipine, nimodipine, phenoxybenzamine, prazosin, reserpine, and terazosin or combinations thereof.

Therapeutic agents include immunosuppressive agents, miscellaneous therapeutic agents, monoamine oxidase inhibitors, NSAIDs, opiate agonists or opiate partial agonists, such as codeine, dextropropyoxyphene, diamorphine, dihydrocodeine, meptazinol, methadone, morphine, nalbuphine, and pentazocine or combinations thereof.

Therapeutic agents also include respiratory tract agents, skeletal muscle relaxants, thyroid and anti-thyroid agents, such as carbimazole and propylthiouracil; tricyclics and other norepinephrine-reuptake inhibitors, vitamins, wakefulness-promoting agents, sildenafil, tadalafil or combinations thereof.

### Anti-Viral Agents

In particular, the disclosure provides for therapeutic agents is an anti-viral agent. The disclosure provides for methods of treating or alleviating a viral infection. The term “anti-viral agent” refers to an agent that used to inhibit production or replication of viruses that cause disease. Anti-viral agents include agents that inhibit transcription of the viral genome such as DNA polymerase inhibitors and reverse transcriptase inhibitors, protease inhibitors which inhibit post-translational events, agents that inhibit the virus from attaching to or penetrating the host cell. Anti-viral agents include immunomodulators that induce production of host cell enzymes, which stop viral reproduction, integrase strand transfer inhibitors that prevent integration of the viral DNA into the host DNA by inhibiting the viral enzyme integrase, and neuraminidase inhibitors that block viral enzymes and inhibit reproduction of the viruses.

Exemplary anti-viral agents include adamantane antivirals, antiviral boosters, antiviral combinations, antiviral interferons, chemokine receptor antagonist such as CCR5-antagonists, integrase strand transfer inhibitor, miscellaneous antivirals, neuraminidase inhibitors, NNRTIs, NS5A inhibitors, fusion inhibitors, nucleoside reverse transcriptase inhibitors (NRTIs), protease inhibitors, guanosine analog, DNA polymerase inhibitors, guanine nucleotide synthesis inhibitors, and purine nucleosides.

The disclosure provides for methods of treating respiratory disorders caused by respiratory viruses include the influenza viruses (A and B), H5N1 and H7N9 avian influenza A viruses, parainfluenza viruses 1 through 4, adenoviruses, respiratory syncytial virus A and B and human metapneumovirus, and rhinoviruses (see Table: Some Respiratory Viruses), coronavirus such as Middle East respiratory syndrome coronavirus (MERS-CoV), severe acute respiratory syndrome coronavirus SARS-CoV, and severe acute respiratory syndrome coronavirus 2 (SARS-CoV2). The respiratory disorders include influenza, common cold, MERS, SARS and COVID-19, Influenza, AFRD, acute bronchitis and pneumonia, and croup, to name a few.

The disclosure provides for methods of treating gastroenteritis caused by a virus including Rotavirus, Norovirus, astrovirus, adenovirus 40, adenovirus 41, and coronavirus, to name a few.

The disclosure provides for methods of treating an infection or disorder caused by Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), or coxsackievirus.

Viral infections that can be treated include, at least, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis (encephalitis) (VEE) virus, Chikungunya virus, Western Equine Encephalomyelitis (encephalitis) (WEE) virus, Eastern Equine Encephalomyelitis (encephalitis) (EEE) virus, Tick-borne Encephalitis, Kyasanur Forest Disease, Alkhurma Disease, Omsk Hemorrhagic Fever, Hendra virus, Nipah virus, and species thereof.

In a particular aspect of the disclosure provides for methods of treatment and prophylaxis of herpes infections. e.g. HSV-1 and HSV-2, in particular Herpes simplex infections in patients displaying Herpes labialis, Herpes genitalis, Herpetic gingivostomatitis and Herpes-related keratitis, Alzheimers disease, encephalitis, pneumonia, hepatitis; dermatitis, keratoconjuctivitis, Vulvovaginitis, in patients with a suppressed immune system, such as AIDS patients, cancer patients, patients having a genetic immunodeficiency, transplant patients; in new-born children and infants; in Herpes-positive patients, in particular Herpes-simplex-positive patients, for suppressing recurrence (suppression therapy); patients, in particular in Herpes-positive patients, in particular Herpes-simplex-positive patients.

The disclosure also provides for methods of treating infections caused by human papillomavirus, and the disorders caused by such as warts (verrucae), genital warts, cervical cancer, anogenital cancer and oropharyngeal cancer.

In addition, the methods include treating infections caused by Additional virus include Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses.

The disclosure also includes methods of treating an infection caused Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV, Venezuelan equine encephalomyelitis VEE.

The methods of treating a viral infection include methods which reduce of viral load or prevent the viral load from increasing. In particular, the methods reduce viral replication or neutralize the virus. In this way, the disclosed methods prevent the viral load from increasing to a point where it could cause pathogenesis, allowing the body's innate immune mechanisms to neutralize the virus.

Exemplary anti-viral agents include maraviroc, enfuvirtide, amantadine, lamivudine, nevirapine, efavirenz, dolutegravir, elvitegravir, raltegravir, acyclovir and any nucleoside analog of aciclovir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine and sofobuvir. Other exemplary anti-viral agents include peptide based compounds that can target viral/non-viral targets and DNA based compounds that can target viral/non-viral targets.

### Antibacterial Agents

Exemplary antibacterial agents include sulphonamides, amphenicols such as chlorophenicols, spectinomycin, trimethoprim, tigencycline, erythromycin, clarithromucin, azithromycin, linezolid, deoxyclcline, carbapenems such as imipenem, meropenem, aztreonam, ticaracillinclvulnate, piperaciin-tazobactam, cephalosporin, e.g. cefotaxime, ceftriaxone, ceftazidime, and cefepime, gentamicin, tobramycin, and amikacin, Quinolones such as axaquin (lomefloxacin), Floxin (ofloxacin), Noroxin (norfloxacin), Tequin (gatifloxacin), Cipro (ciprofloxacin), Avelox (moxifloxacin), Levaquin (levofloxacin), Factive (gemifloxacin), Cinobac (cinoxacin), NegGram (nalidixic acid), Trovan (trovafloxacin), and Zagam (sparfloxacin), flouroquinolones, such as levofloxacin, ciprofloxacin, and oxifloxacin, vancomycine, polymyxins such as polymyxin B and colistin, cephalosporins, carbepenems, macrolides such as axaquin (lomefloxacin), Floxin (ofloxacin), Noroxin (norfloxacin), Tequin (gatifloxacin), Cipro (ciprofloxacin), Avelox (moxifloxacin), Levaquin (levofloxacin), Factive (gemifloxacin), Cinobac (cinoxacin), NegGram (nalidixic acid), Trovan (trovafloxacin), and Zagam (sparfloxacin), Vancomycin, clindamycin, isoniazid, rifampin, ethambutol, pyrazinamide, bacitracin, polymixins, sulfonamides, glycopeptide and nitroimidazoles, Ticarcillin, a carboxypenicillin, Chloramphenicol, Rifamycins, and penicillins, e.g. penicillin V, penicillin G, procaine penicillin G, benzathine penicillin G, methicillin, oxacillin, cloxacillin, dicloxacillin and flucloxacillin, ampicillin, amoxicillin, propicillin, pheneticillin, azidocillin, clometocillin, and penamecillin or combinations thereof.

### Cytokines and Growth Factors

Therapeutic agents include cytokines and growth factors, such as those that are effective in inhibiting tumor metastasis. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 a, cytokine-induced neutrophil chemotactic factor 2β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type 1, tumor necrosis factor receptor type II, urokinase-type plasminogen activator receptor, and chimeric proteins and biologically or immunologically active fragments thereof or combinations thereof.

### Anticancer Agents

Therapeutic agents include cytotoxic agents. The cytotoxic agent is any molecule (chemical or biochemical) which is toxic to a cell. In some embodiments, the cytotoxic agent is a chemotherapeutic agent. Chemotherapeutic agents are known in the art and include, but not limited to, platinum coordination compounds, topoisomerase inhibitors such as a camptothecin or a camptothecin analog, antibiotics, antimitotic alkaloids and difluoronucleosides, as described in U.S. Pat. No. 6,630,124.

In some embodiments, the chemotherapeutic agent is a platinum coordination compound. The term “platinum coordination compound” refers to any tumor cell growth inhibiting platinum coordination compound that provides the platinum in the form of an ion. In some embodiments, the platinum coordination compound is Cisplatin, or cis-dichlorodiammineplatinum II, cis-diamminediaquoplatinum (II)-ion; chloro(diethylenetriamine)-platinum(II)chloride; dichloro(ethylenediamine)-platinum(II), diammine(1,1-cyclobutanedicarboxylato) platinum(II) (carboplatin); spiroplatin; iproplatin; diammine(2-ethylmalonato)-platinum(II); ethylenediaminemalonatoplatinum(II); aqua(1,2-diaminodyclohexane)-sulfatoplatinum(II); (1,2-diaminocyclohexane)malonatoplatinum(II); (4-caroxyphthalato)(1,2-diaminocyclohexane)platinum(II); (1,2-diaminocyclohexane)-(isocitrato)platinum(II); (1,2-diaminocyclohexane)cis(pyruvato)platinum(II); (1,2-diaminocyclohexane)oxalatoplatinum(II); ormaplatin; tetraplatin, diamino-platinum complexes include, such as spiroplatinum and carboplatinum

In some aspects, the therapeutic agent is a topoisomerase inhibitor is camptothecin or a camptothecin analog. Camptothecin is a water-insoluble, cytotoxic alkaloid produced by Camptotheca accuminata trees indigenous to China and Nothapodytes foetida trees indigenous to India. Camptothecin exhibits tumor cell growth inhibiting activity against a number of tumor cells. Compounds of the camptothecin analog class are typically specific inhibitors of DNA topoisomerase I. By the term “inhibitor of topoisomerase” is meant any tumor cell growth inhibiting compound that is structurally related to camptothecin. Compounds of the camptothecin analog class include, but are not limited to; topotecan, irinotecan and 9-amino-camptothecin.

In additional embodiments, the cytotoxic agent is any tumor cell growth inhibiting camptothecin analog claimed or described in: U.S. Pat. No. 5,004,758, issued on Apr. 2, 1991 and European Patent Application Number 88311366.4, published on Jun. 21, 1989 as 20′ Publication Number EP 0 321 122; U.S. Pat. No. 4,604,463, issued on Aug. 5, 1986 and European Patent Application Publication Number EP 0 137 145, published on Apr. 17, 1985; U.S. Pat. No. 4,473,692, issued on Sep. 25, 1984 and European Patent Application Publication Number EP 0 074 256, published on Mar. 16, 1983; U.S. Pat. No. 4,545,880, issued on Oct. 8, 1985 and European Patent Application Publication Number EP 0 074 256, published on Mar. 16, 1983; European Patent Application Publication Number EP 0 088 642, published on Sep. 14, 1983; Wani et al., J. Med. Chem., 29, 2358-2363 (1986); Nitta et al., Proc. 14th International Congr. Chemotherapy, Kyoto, 1985, Tokyo Press, Anticancer Section 1, p. 28-30, especially a compound called CPT-11. CPT-11 is a camptothecin analog with a 4-(piperidino)-piperidine side chain joined through a carbamate linkage at C-10 of 10-hydroxy-7-ethyl camptothecin. CPT-11 is currently undergoing human clinical trials and is also referred to as irinotecan; Wani et al, J. Med. Chem., 23, 554 (1980); Wani et. al., J. Med. Chem., 30, 1774 (1987); U.S. Pat. No. 4,342,776, issued on Aug. 3, 1982; U.S. patent application Ser. No. 581,916, filed on Sep. 13, 1990 and European Patent Application Publication Number EP 418 099, published on Mar. 20, 1991; U.S. Pat. No. 4,513,138, issued on Apr. 23, 1985 and European Patent Application Publication Number EP 0 074 770, published on Mar. 23, 1983; U.S. Pat. No. 4,399,276, issued on Aug. 16, 1983 and European Patent Application Publication Number 0 056 692, published on Jul. 28, 1982; the entire disclosure of each of which is hereby incorporated by reference. All of the above-listed compounds of the camptothecin analog class are available commercially and/or can be prepared by conventional techniques including those described in the above-listed references. The topoisomerase inhibitor may be selected from the group consisting of topotecan, irinotecan and 9-aminocamptothecin.

The preparation of numerous compounds of the camptothecin analog class (including pharmaceutically acceptable salts, hydrates and solvates thereof) as well as the preparation of oral and parenteral pharmaceutical compositions comprising such a compounds of the camptothecin analog class and an inert, pharmaceutically acceptable carrier or diluent, is extensively described in U.S. Pat. No. 5,004,758, issued on Apr. 2, 1991 and European Patent Application Number 88311366.4, published on Jun. 21, 1989 as Publication Number EP 0 321 122, the teachings of which are incorporated herein by reference.

In still yet other embodiments of the disclosure, the chemotherapeutic agent is an antibiotic compound. Suitable antibiotic include, but are not limited to, doxorubicin, mitomycin, bleomycin, daunorubicin and streptozocin.

In some embodiments, the chemotherapeutic agent is antimitotic alkaloids of the present disclosure include, but are not limited to, vinblastine, vincristine, vindesine, Taxol and vinorelbine. In other embodiments, the chemotherapeutic agent is a difluoronucleoside such as 2′-deoxy-2′,2′-difluoronucleosides or 2′-deoxy-2′,2′-difluorocytidine hydrochloride, also known as gemcitabine hydrochloride.

### EXAMPLES

### Example 1

### Materials and Methods

HPAC, produced via carbonization of coconut shell at 1300° C., in the size range of 1 μm were procured from US Research Nanomaterials, Inc, Texas, USA. Accordingly, 10 mg/mL and 1 mg/mL aqueous solutions were prepared by mixing required amounts of HPAC to sterile PBS. These HPAC powders are reported to have an Iodine Adsorption of 1380 mg/g and Specific surface area of 1360 m²/g. Acyclovir was purchased from Selleckchem at 99% purity (50 mg) and used at a dilution of 50 mM in DMSO.

### Cells and Viruses

Chinese hamster ovary (CHO-K1) cells were passaged in Ham's F12 medium (Gibco/BRL, Carlsbad, Calif., USA) supplemented with 10% fetal bovine serum (FBS) and 1% penicillin and streptomycin (P/S) (Sigma). HeLa cells were passaged in Dulbecco's modified Eagle's medium (DMEM) supplemented with 10% FBS and 1% P/S. African green monkey kidney (Vero) cells, human vaginal epithelial (VK2/E6) cells and human foreskin fibroblasts (HFFs) cells were passaged in Dulbecco's modified Eagle's medium (DMEM) supplemented with 10% FBS and P/S. The human corneal epithelial cell line (HCE) (RCB1834 HCE-T) was obtained from Kozaburo Hayashi (National Eye Institute) and passaged in minimum essential medium (MEM) (Gibco/BRL, Carlsbad, Calif., USA) supplemented with 10% FBS and 1% P/S.

The following viral strains were used in the studies described below: HSV-1 (17 GFP), HSV-2 (333 strain), HSV-2 GFP (333 strain—GFP variant), and β-galactosidase-expressing HSV-1 (gL86), HSV-2 (333)gJ, PRV and BHV. Virus stocks were propagated and tittered on Vero cells, and stored at −80° C.

### MTT Assay

MTT (3-(4,5-dimethylthiazol-2-yl)-2,5-diphenyltetrazolium bromide) viability assay on HCE VK2, HFF and HeLa cell lines using various concentrations of HPAC were performed post 24 hour incubation. Briefly, cells were plated at a density of 1×10⁴ per well in a 96 well plate overnight. Following morning, concentrations starting at 10 mg/mL were two-fold serially diluted and added to cell monolayers in whole media for a period of 24 h. At the end of incubation, 0.5 mg/mL in whole media) was added to cells and incubated for a period of 3 hours to allow crystal formation. Acidified isopropanol (1% glacial acetic acid v/v) was added to cells to dissolve the formazan crystals. Dissolved violet crystals were transferred to a new 96 well plate and analyzed by a micro-pate reader (TECAN GENious Pro) at 492 nm. MTT assay was conducted on cell-free HPAC conditions and their values were subtracted from the cell-mediated conditions to account for signal generated through oxidative degradation of MTT by HPAC alone.

### Treatment Models

HPAC was dispersed in Opti-MEM at 10 mg/mL concentration and used as stock for all the experiments. DECON was dispersed in Opti-MEM at 1 mg/mL concentration and used as stock for all the experiments.

Prophylaxis: HPAC or DECON were added to monolayer of cells at required concentration 30 minutes prior to infection. Cell monolayer was washed twice with PBS before whole media with required HSV infection was added.

Neutralization: HPAC or mock at required concentration was incubated with HSV-1 or HSV-2 for a period of 30 minutes in an Eppendorf tube and mixed continuously in a tube holder before the contents were centrifuged at 10,000 g. The supernatant was carefully removed without disturbing the pellet and added on to a monolayer of cells.

### Viral Entry Assay

3-Galactosidase expressing viruses HSV-1 gL 86 and HSV-2 (333)gJ—at MOI of 10 were used in this study. Cells were plated at a density of 1×10⁴ in 96 well plates overnight before use. Neutralization or prophylactic treatment using HPAC at concentrations of 1, 0.5 and 0.1 mg/mL were conducted before the cells were infected with the virus at 37° C. HSV-1 strain gL86 and HSV-2 strain gJ—were allowed to infect cell monolayers for 6 h after which the cells were washed with PBS twice and 100 μL of soluble substrate o-nitrophenyl-3-d-galactopyranoside (ONPG) 3 mg/mL was added to the cells along with 0.5% Nonidet P-40 in PBS. Enzymatic activity was measured by a micro-pate reader (TECAN GENious Pro) at 405/600 nm.

### Immunoblotting

Cells were scraped and incubated with 100 μL of Radioimmunoprecipitation assay (RIPA) buffer with protease phosphatase inhibitor (Halt™ Protease and Phosphatase Inhibitor Cocktail (100×); Thermofisher) for a period of 30 minutes on ice. Whole cell protein extracts (supernatant) were collected by centrifuging the mixture at 13,500 rpm on a bench top refrigerated (4° C.) centrifuge for 15 minutes. Protein samples were then denatured in NuPAGE LDS Sample Buffer (Invitrogen, NP00007) and 3-mercaptoethanol by heating them to 80° C. for 10 min. The denatured protein samples were allowed to cool and equal amounts of protein were added to 4-12% SDS-PAGE loading gels and run at a constant speed of 70V for 3 h. The protein from the gel was then transferred to a nitrocellulose membrane using an iBlot 2 dry transfer instrument (Thermofisher Scientific, USA). Nitrocellulose membrane was blocked in 5% nonfat milk in tris buffer saline (TBS) and 0.1% tween 20 (TBST) for 1 h at room temperature. After the blocking step, membranes were incubated with anti-HSV-1 and HSV-2 gB mouse monoclonal antibody (abcam, 6506) or anti-GAPDH (Proteintech, 10494-1-AP) antibody at dilutions of 1:1000 overnight at 4° C. The following day the blots were washed multiple times with TBST before the addition of horse radish peroxidase conjugated secondary IgG antibody at dilutions of 1:10,000 at room temperature. Protein bands were visualized on an ImageQuant LAS 4000 imager (GE Healthcare Life Sciences) by the addition of SuperSignal West Pico maximum sensitivity substrate (Pierce, 34080). The density of the bands was quantified using ImageQuant TL image analysis software (version:7). GAPDH was measured as a loading control.

### Plaque Assay

Cells infected with virus mentioned in the previous section were suspended in 500 μL of Opti-MEM and sonicated using a probe sonication system at 70% amplitude for 30 seconds. These sonicated used as inoculants for a plaque assay to determine the total amount of infectious viruses released from the infected cells. In a typical experiment, Vero cells were plated at a seating density of 5×10⁴ per well in a 24 well plate. Upon confluency, the cell monolayers were washed with PBS and supernatants (mentioned above) were used as inoculants of infection for a period of 2 h. After completion of 2 h, the cells were washed twice with PBS and DMEM mixed with 0.5% methylcellulose was added. The plates were incubated for 72 h at 37° C. and 5% C02 before they were fixed with methanol and stained with crystal violet to determine the extent of plaque formation. Plaques were counted using a 2× microscope and total plaque count was tallied with each other to determine the efficacy of AC treatment.

### Quantitative PCR Assay

RNA from cells was extracted using Trizol (Life Technologies) according to manufacturer's described protocol. Thus obtained RNA was quantified using Nanodrop (Thermofisher Scientific, USA) and equilibrated for all samples with Molecular Biology Grade water (Corning, USA) before they were reverse transcribed into cDNA using High Capacity cDNA Reverse Transcription Kit (Applied Biosystems, Foster City, Calif.). Equal amounts of cDNA were analyzed via real-time quantitative PCR using Fast SYBR Green Master Mix on QuantStudio 7 Flex system (Applied Biosystems). The primers used in this study are as follows:

| | |
|---|---|
| GAPDH fwd 5′-TCCACTGGCGTCTTCACC-3′ | (SEQ ID NO: 1) |
| and | |
| rev 5′-GGCAGAGATGATGACCCTTTT-3′ | (SEQ ID NO: 2) |
| IFNα fwd 5′-GATGGCAACCAGTTCCAG | (SEQ ID NO: 3) |
| AAG-3′; | |
| IFNα rev 5′-AAAGAGGTTGAAGATCTG | (SEQ ID NO: 4) |
| CTGGAT-3′; | |
| IFNf3 fwd 5′-CTCCACTACAGCTCTTT | (SEQ ID NO: 5) |
| CCAT-3′; | |
| IFNf3 rev 5′-GTCAAAGTTCATCCTGT | (SEQ ID NO: 6) |
| CCTT-3′; | |
| TNFα fwd 5′-AGCCCATGTTGTAGCAAA | (SEQ ID NO: 7) |
| CCC-3′; | |
| TNFα rev 5′-GGACCTGGGAGTAGATGA | (SEQ ID NO: 8) |
| GGT-3′. | |

### Florescent Virus Infection Model

Mock, neutralization and prophylactic treatments similar to those mentioned in earlier sections were conducted with an MOI of 50 using GFP fluorescent viruses. The infection was allowed to occur for a period of 90 minutes at 4° C. followed by a brief incubation of 15 minutes at 37° C. This process allowed for the virus to remain on the cell surface rather than to enter the cell. After 15 minutes of incubation, the cells were fixed using 4% paraformaldehyde followed by nuclear (DAPI) and actin (rhodamine-phalloidin) staining. The cells were visualized to understand the extent of infection reaching the cell surface. Five images per sample were procured and quantitatively analyzed for GFP fluorescence per image. The quantitative data would directly translate to the total amount of viral infection in a given area. The fluorescent intensity of the all the channels were kept constant and the images were analyzed using MetaMorph Microscopy Automation and Image Analysis Software to quantitatively determine total intensity from the green channel.

### Flow Cytometry

Cells infected with GFP virus were collected 24 hpi using Hank's based, enzyme-free cell dissociation buffer (Gibco-13150). Cells were washed once with PBS before they were fixed using 4% parafolmaldehyde (Electron Microscopy Sciences, USA) and suspended in fluorescence-activated cell sorting (FACS) buffer (PBS, 5% FBS). Cell suspensions were filtered through a 70-μm mesh, resuspended in FACS buffer before analyzing them using an in-house flow cytometer (BD Accuri C6 Plus). A total of 4×10⁴ cells were collected for each sample and the analysis was performed using FlowJo (version 10).

### ACV Standard Curve

Increasing amounts of ACV (Selleckchem, USA) dissolved in DMSO was suspended in PBS to make a concentration gradient. NanoDrop UV Spectrophotometer was used to determine the absorption peak of ACV at 252 nm. Typically 2 μL of desired solution was dropped on to the analysis port of the NanoDrop before closing the lid and starting the measurement. Each measurement was conducted thrice with 3 individual drops and three separate replicates were used to determine the absorbance of every ACV concentration. The readings were noted down manually and entered into GraphPad Prism software to prepare the concentration vs absorbance curve.

### Drug Loading Studies

100 μg of ACV, dissolved in 50 μL of DMSO was added to 1 mg/mL HPAC and incubated at room temperature overnight on a 3D rocker (BioRocker 3D, Denville, USA). The mixture was centrifuged at 14,000 g for 15 minutes to separate the drug loaded HPAC particles from the free drug supernatant. Supernatant was analyzed for the amount of ACV using the UV. spectrophotometer as mentioned above. Three individual loading experiments were conducted to analyze the drug loading efficiency and as prepared DECON particles were stored at 4° C. for further characterization.

### Drug Release Studies

Passive release: Seven individual vials containing 1 mg/mL DECON particles diluted in 1 mL MEM media were incubated at 37° C. for up to 7 days. Every day, a single vial was removed and centrifuged at 14,000 g for 15 minutes. 2 μL of the supernatant was used to analyze the ACV concentration using a UV Spectrophotometer as mentioned above. MEM media was used as blank for these studies.

Triggered release: For active drug release studies, purified virus (sucrose gradient purification) and cell debris (50 million HCEs sonicated in 1 mL MEM media) were used. Increasing concentrations of purified virus and cell debris was added to DECON (1 mg/mL) in MEM media and incubated for a period of 15 minutes at 37° C. before the mixtures were spun at 14,000 g and the supernatants were analyzed using the UV spectrophotometer for the presence of ACV. In parallel, DECON (1 mg/mL in MEM) was heated to 90° C. using a pre-heated heat block for period of 5 minutes before the samples were centrifuged and supernatant analyzed for ACV. Also, DECON (1 mg/mL in MEM) was sonicated using a probe sonicator (Fisher Scientific, USA) at 30% amplitude for 40 seconds separated by a 5 second pause. The sample was then centrifuged at 14,000 g before the supernatant was analyzed for the amount of ACV using a UV spectrophotometer.

Sustained release: 5×10⁴ PFU of purified virus or equivalent cell debris was added to 100 μL of DECON (1 mg/mL in MEM) and incubated at 37° C. for a period of 24 hours. At set time intervals, 2 μL of supernatant from the vial was removed and analyzed for the concentration of ACV using a UV spectrophotometer.

### In Vivo Ocular HSV-1 Infection and Treatment

C57BL/6 mice, bred and housed at the university biological resource laboratory (BRL) were used for ocular model of murine HSV-1 infection. Standard feed and water were provided to the mice with a 12 h light and dark cycle with no more than 5 mice per cage. On day 0, 6 to 8 week old mice were anesthetized, as described previously, prior to the application of a topical anesthetic (proparicaine hydrochloride, 0.5%). Corneal epithelial debridement was performed using a 30 G needle followed by the application of 5×105 PFU HSV-1 (McKrae) to the eye. Topical treatments were performed on days 1, 3, 5, 7 and 9 post infection, while ocular washes and mice pictures (Carl Zeiss Stereoscope) were collected on days 2, 4, 7 and 10 post infection.

### Corneal Sensitivity Recording

Corneal sensitivity of the mice eyes was measured by manual Aesthesiometer (12/100 mm, LUNEAU SAS, France). The aesthesiometer consisting of a nylon filament 6 cm in length is applied to the center of the mouse cornea and the pressure exerted was measured blink response. At the highest length, if the mice blinked, it was considered most responsive and the absence of a blink at the shortest length was considered least responsive. The length of the nylon filament was reduced 0.5 cm at a time to record the blink response. The measurement was taken in triplicates at 10 sec intervals. The mice that did not respond were given an arbitrary score of 0. High score indicates high/normal corneal sensitivity, while a low score corresponds to the absence or loss of corneal sensitivity.

### In Vivo Vaginal Infection and Treatment

Naïve 4-week-old female BALB/c mice were purchased from Jackson Laboratory (Bar Harbor, USA) and housed in the university BRL for a period of one week for acclimatization before they were subcutaneously injected with 2 mg of medroxyprogesterone (Depo-Provera) per mice. On day 5 post injection, mice were intravaginally infected with 1×106 PFU HSV-2 (333 strain). Similar to the ocular model of infection, DECON was applied topically (intravaginally) on alternate days while ACV (50 mg/mL in PBS) was administered via an intraperitoneal injection. PBS was applied intravaginally as mock treatment for the control group. Vaginal swabs were collected using a Calgiswab (Calcium Alginate Mini-tip Urethro-Genital Swab, Puritan) dipped in OptiMEM (Gibco, USA). Images of the ano-genital region were taken on day 0 and day 7 post infection using a Carl Zeiss stereoscope at 7.5× magnification.

Experiments involving animals were performed under a UIC approved protocol ACC 14-091. The mice were monitored for weight loss and disease scores were recorded in a blinded fashion for 14 days. Sick mice were euthanized according to the IUCAC protocol followed by the collection of ocular/vaginal tissue and lymph nodes. Ocular wash and vaginal swabs were used to assess viral titers using a plaque assay.

### Statistical Analysis

Graph Pad Prism software (version 4.0) was used for statistical analysis of each group. P<0.05 and P<0.001 were considered as the significant differences among mock treated and treated groups.

### Example 2

### Prophylactic, Neutralization and Therapeutic Efficacy of HPAC

To quantify the virostatic potential of HPAC, a reporter-based virus entry assay was conducted using a prophylactic treatment model. Hela or HCE cells were prophylactically treated for 90 minutes with HPAC, followed by HSV-1 or HSV-2 infection at a multiplicity of infection (MOI) of 10 for 6 hours.

HPAC Inhibits HSV from Entering into Cells

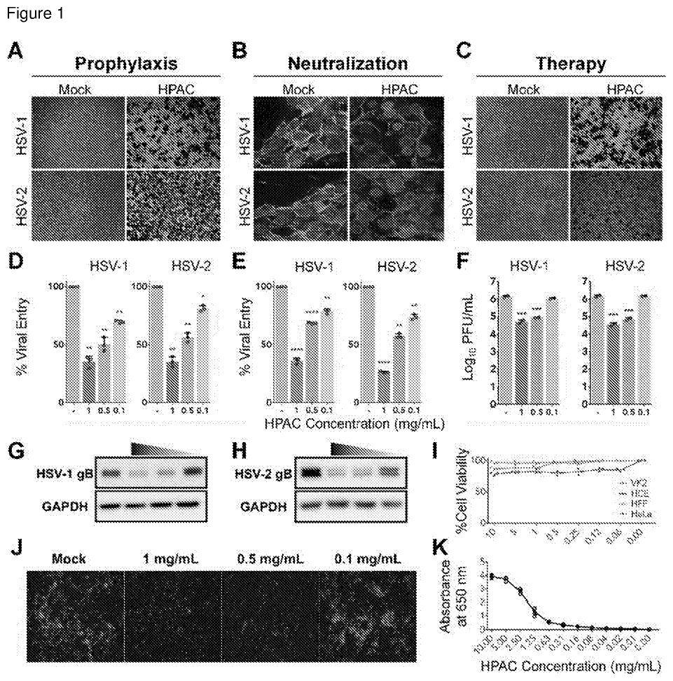

FIG. 1A provides fluorescence imaging of GFP-HSV-1 and GFP-HSV-2 infected HCE or HeLa cells treated with 1 mg/mL HPAC prophylactically. A 40 to 60% reduction in HSV-1 and HSV-2 entry was shown using concentrations of HPAC as low as 1 mg/mL (FIGS. 1A and 1D). The inhibitory concentration 50 (IC50) value of HPAC during HSV-1 and HSV-2 infection in prophylactic treatment was found to be 0.8 mg/mL and 1 mg/mL respectively. These values are substantially below the clinically accepted TC50 value of HPAC (>50 mg/mL), making them a viable material for inhibiting virus entry. The selectivity index (SI=TC50/IC50) for HPAC in prophylactic model for HSV-1 and HSV-2 were calculated as 62.5 and 50 respectively.

### HPAC Traps HSV Particles

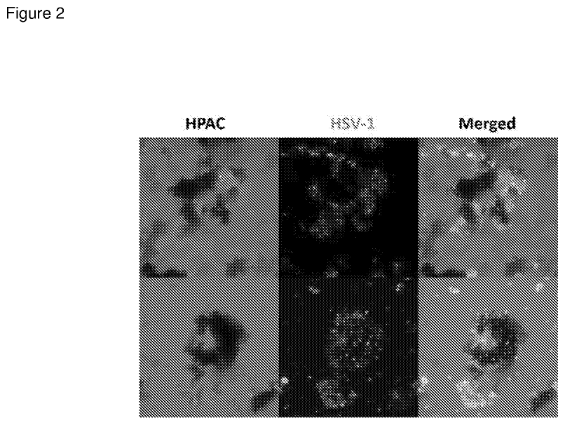

To determine the mechanism behind the virostatic potential of the HPAC on HSV-1 and HSV-2, GFP virus was incubated with HPAC for a period of 30 minutes. Following incubation, HPAC was separated from non-neutralized virus in the supernatant and overlaid on HeLa cells. 1 mg/mL HPAC was incubated with 106 PFU K26 GFP HSV-1 for a period of 20 minutes before the mixture was centrifuged at 10,000 g for 15 minutes. The pellet containing HPAC was washed multiple times with PBS before the mixture was suspended in fresh PBS. 10 μL of the mixture was dropped onto a glass slide. Six hours post infection, centrifuged HPAC pellet (shown in FIG. 2) and HeLa cells infected with supernatant virus (FIG. 1c—right) were fixed and imaged.

Given the highly porous and active nature of HPAC's surface, it was hypothesized that it would be able to efficiently trap the virus in its nanopores. As expected, HPAC neutralization contained significantly lower virus compared to mock neutralized virus. In addition, an entry assay was performed similar to one explained in the previous section using a β-galactosidase reporter virus. The results from the entry assay confirmed that HPAC efficiently bound the virus and restricted viral entry into cells. This confirms that HPAC at a concentration of 1 mg/mL inhibits virus entry by trapping the virus in its nanopores, thereby neutralizing the virus prior to its application to HeLA cells (FIGS. 1b and 1e). The cells were stained using DAPI staining for DNA, Phalloidin for actin and GFP. Viral entry was measured using the a β-galactosidase reporter virus.

### HPAC Treatment Shows Therapeutic Efficacy

To further investigate the antiviral potential of HPAC treatment, HCEs and HeLa cells were first infected with HSV-1 and HSV-2 respectively for a period of 2 hours before the addition of Mock PBS or HPAC at 1 mg/mL (0.1 MOI) then therapeutically treated with HPAC. Cells were imaged (FIG. 1c) and the cell lysates were collected from these samples at 24 hpi were used as inoculants for plaque assays to determine intracellular viral load. Fluorescence images were taken to understand the extent of viral spread in HPAC treated samples compared to mock. Intracellular viral load for HPAC therapeutic treatment was quantified using a plaque assay. The number of observed plaques for HPAC-treated samples were significantly fewer than mock-treated samples. (FIG. 1f). Representative immunoblots of HSV-1 as shown in FIG. 1G and HSV-2 gB in FIG. 1H, human glyceraldehyde-3-phosphate dehydrogenase (GAPDH).

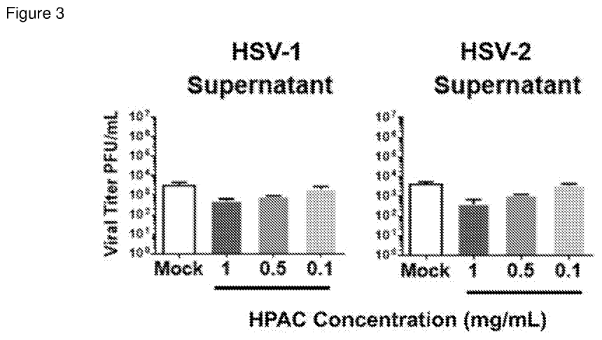

Supernatants collected from this experiment were used to determine the released virus load. As shown in FIG. 3, HCEs and HeLa cells were infected with HSV-1 or HSV-2 respectively for 2 hours before difference concentrations of mock PBS or HPAC was therapeutically added. 24 hours post infection, supernatants from the treated samples were collected and overlaid on Vero cells to perform an extracellular virus-based plaque assay. Immunoblotting for the presence of HSV-1 and HSV-2 gB (viral protein) indicated a significant reduction (>50%) in gB bands when cells were treated with 0.5 mg/mL HPAC compared to mock-treated controls (FIG. 1g). Furthermore, the same experimental set up was implemented again, this time using the cell lysates.

### HPAC Blocks HSV-Induced Syncytia Formation

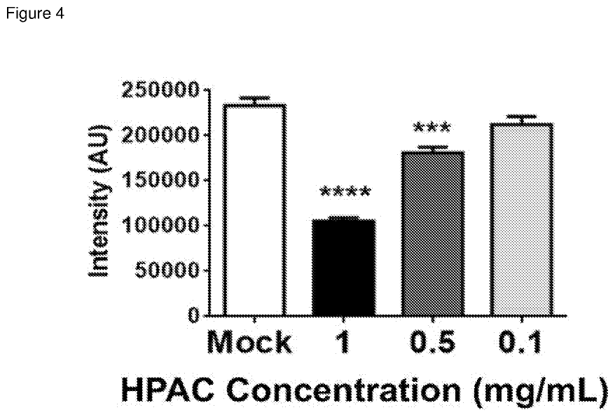

It is well known that HSV-1 and HSV-2 viruses spread from one cell to another through the formation of syncytial structures. Given that HPAC strongly binds to the cell surface and the significant reduction in viral spread observed, it was hypothesized that HPAC may be inhibiting syncytia formation and therefore resulting in reduced viral spread. To test this, a virus-free transfection-based assay was used to determine syncytia formation. Plasmids of viral glycoprotein gB, gD, gH and gL along with T7 polymerase were transfected into Chinese hamster ovary (CHO) cells and termed as ‘effector cells’. Similarly, plasmids of Nectin-1 and T7 luciferase promoter were transfected into a different set of CHO cells and termed as ‘target cells’. When both the ‘effector’ and ‘target’ cells are combined in vitro, they form syncytial structures similar to those observed during HSV infection. This was quantitatively analyzed by measuring the luciferase activity produced as a result of syncytia structure formation. This assay was conducted in the presence of HPAC at varying concentrations to see its effect on syncytia formation. First, HPAC was added to ‘target cells’. ‘Effector’ cells were then added to the ‘target’ cells and incubated for 24 h. The cells were imaged and quantitatively analyzed for syncytial structures and luciferase activity (FIG. 1j and FIG. 4). It was observed that HPAC successfully reduced syncytial structure formation at 1 and 0.5 mg/mL concentrations, confirming the hypothesis.

### Example 3

### HPAC is Biocompatible and Non-Toxic

In order to determine the viability of cells in the presence of HPAC, an MTT assay was conducted on human corneal epithelial cells (HCEs), human foreskin fibroblasts (HFFs), vaginal epithelial cells (VK2s) and HeLa cell lines. Evidently, no significant toxic effect or loss in cell viability was observed when HPAC was used at concentrations as high as 10 mg/mL (FIG. 1i). All the concentrations used showed cell viability greater than 75%, proving no cytotoxic effects in HCEs, HFFs, VK2s and HeLa cell lines when HPAC is used at a size of about 1 μm.

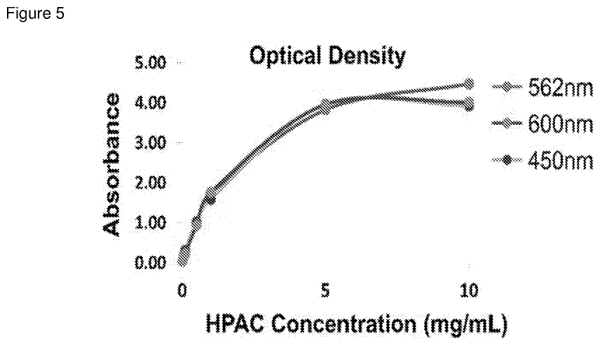

Furthermore, the optical densities of HPAC at various concentrations were determined using a standard plate reader for absorption at 650 nm. The optical density of HPAC was near zero under the concentration of 0.5 mg/mL (FIG. 1k). The optical density measured at other wavelengths (450, 562 and 600 nm) gave similar absorbance values (FIG. 5).

HPAC Treatment does not Induce Host Cytokine Response

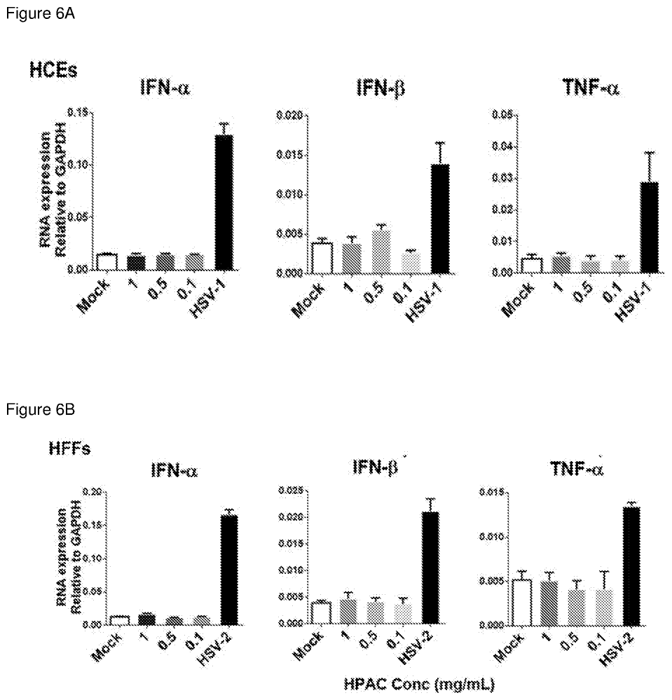

While the virostatic potential of HPAC was evident from entry assays, its effect on host cytokine response was also investigated. Previous studies on nanoparticles have shown that their prolonged surface attachment could instigate cytokine production and in-turn create disease-like effects. In addition, many nanoparticle and micro-particle based antivirals inhibit viral replication/entry through upregulation of cytokines such as interferon-alpha (IFN-α), interferon-beta (IFN-β) and tumor necrosis actor-alpha (TNF-α). To evaluate this potential effect with HPAC, these common host-triggered antiviral markers were measured using quantitative RT-PCR. HCEs and HFFs treated with HPAC for a period of 24 h did not elicit such response when compared to mock-treated or HSV-1/2 infected samples (FIG. 6A and FIG. 6B). While these results do not support the notion that HPAC treatment elevates host immune response, it does indicate that repetitive HPAC application does not result in interferon elevation.

### Example 4

### HPAV Drug Loading and Sustained Release

HPAC Encapsulates Drugs with High Loading Efficiency

Next, studies were carried out to determine whether HPAC particles improves efficacy or generates synergy when combined with an FDA approved treatment such as ACV. Given the surface active properties, HPAC particles (1 mg/mL) were incubated with acyclovir (100 μg) solution for a period of 12 h to enable drug loading. At the end of incubation, HPAC particles were centrifuged and the loading concentration was determined via the following standard formula:

Loaded Drug=Total Drug−Remaining Drug

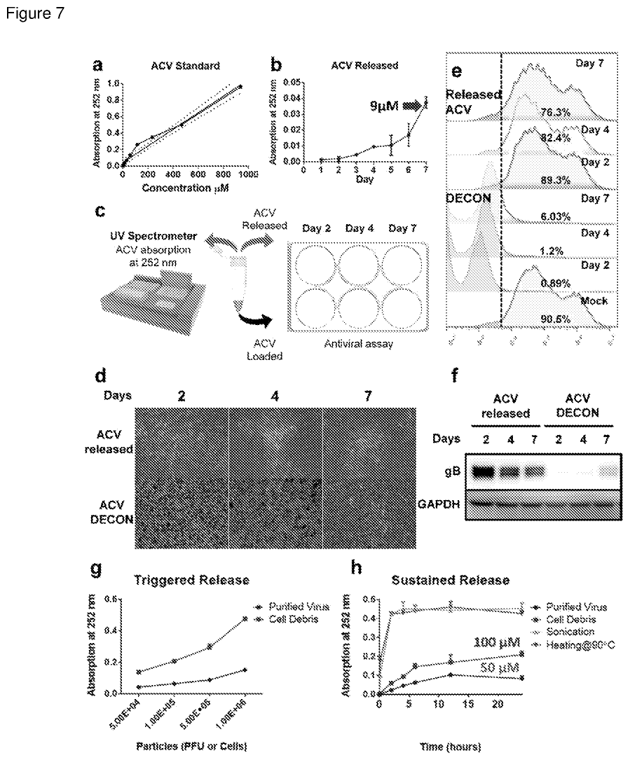

Unexpectedly, greater than 99% drug loading of HPAC was observed when incubated with A. FIG. 7A provides a standard curve generated by UC absorbance at 252 nm. Negligible amount of ACV was detected in the supernatant of the HPAC-drug mix indicating that almost 100% of ACV was loaded in to the HPAC particles forming a DECON particle.

After multiple washes, DECON particles were tested for their drug release characteristics when incubated in PBS or MEM alone. Surprisingly, DECON particles did not release any significant amount of ACV into the surrounding media over an incubation period of 48 hours. To further test sustained drug release, DECON particles were incubated with 1 mL of MEM media for a period of 7 days and samples of the released drug were collected every day via centrifugation (FIG. 7c). Supernatants collected from the samples were analyzed for the presence of ACV followed by their addition to HCEs infected with HSV-1. Similarly, the pellets were resuspended in MEM media and added to HSV-1 infected cells. If a significant amount of ACV was released in to the MEM media over a period of 7 days, there would be a quantifiable antiviral activity associated with the supernatant or the pellet. Unexpectedly, while UV measurement showed only a release of 9 μM ACV in to the media over a period of 7 days (FIG. 7b), the overlaid supernatant showed minimal antiviral activity in HSV-1 infected HCEs (FIG. 7d top, 7e, 7f). Fluorescence staining of either released ACV or ACV loaded DECON taken on either day 2, 4 or 7. 24 hpi, cells were washed, resuspended and fixed with 4% paraformaldehyde before they were analyzed through a BD Accuri C6 flow cytometer as shown in FIG. 7e. Non-infected (GFP-negative) cells were used as negative control and infected-nontreated (GFP-positive) were used as positive control. The panel on the right side indicated in green color represent the number of cells infected in each treatment group. FIG. 7f shows (F) Representative immunoblots from samples treated with Supernatant or DECON pellet on respective days. However the media containing the pellet of DECON particles showed potent antiviral activity (FIG. 7d bottom, 7e, 7f). This was surprising because, although DECON was not passively diffusing ACV into its surrounding media, it showed a potent antiviral activity when added to HSV-1 infected cells.

### Drug Release in DECON is Triggered by the Addition of Virus

As DECON did not passively release drug in to the surrounding medi, studies were carried out to determine whether DECON was interacting with the virus or cellular surface triggering the release of the drug. This series of experiments involved the addition of purified virus and cell debris to DECON. First, increasing amounts of cell debris and purified virus were added to the DECON. Surprisingly, their addition triggered the release of ACV (UV absorption at 252 nm) in a dose dependent manner (FIG. 7g). In Example 2, it was observed that HPAC is able to trap viruses in its pores and attach to cells; that is why the cellular and viral components may be able to competitively bind the surface of DECON dislodging ACV from its pores. In addition, heating DECON to 90° C. for 5 minutes or sonicating it for 20 seconds released most of the ACV (FIG. 7h).

To further investigate whether DECON releases ACV over a period of time in a sustained fashion, we incubated lower concentrations of purified virus and cell debris with DECON for a period of 24 hours. As expected, the addition of virus and cell components was able to release significant amounts of ACV by 24 hours (FIG. 7h). It is also interesting to note that incubation with purified virus triggered the release of approximately 50 μM ACV over a period of 24 hours, a concentration recommended for potent antiviral activity in vitro.

### Example 5

### DECON Particles Restrict HSV Infection In Vitro at Low Concentration

The studies in Example 4 demonstrate that DECON does not release the trapped drug passively; however, it releases ACV in a sustained manner when incubated with virus or cells. To comprehensively test the efficacy of drug release and resulting antiviral activity of DECON particles, a prophylactic and therapeutic model of treatment was used on HCEs followed by HSV-1 infection in vitro.

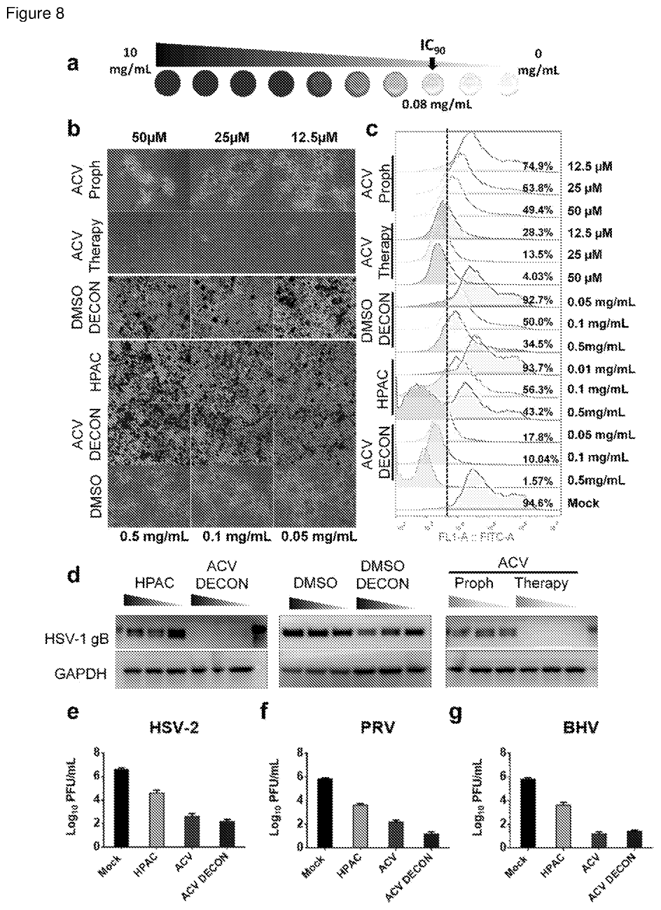

During prophylactic treatment, we observed no viral protein or viral replication in DECON treated cells compared to HPAC alone or mock-treated samples at 0.1 mg/mL concentration (FIGS. 8a, 8b, 8c and 8d). Furthermore, HCEs treated with DECON did not show any signs of toxicity and were well tolerated. The antiviral efficacy of DECON particles was tested at various concentrations starting from 0.05 to 0.5 mg/mL and found that DECON had an IC90 at 0.08 mg/mL (FIG. 8a). FIG. 8b provides fluorescent images showing extent of HSV-1 infection (green) in HCEs treated with either ACV loaded DECON, DMSO loaded DECON, HPAC alone, mock DMSO, prophylactically added ACV or therapeutically added ACV. Flow cytometry was conducted on the samples at 24 hpi showing the extent of cells infected with HSV-1 GFP (see FIG. 8C). FIG. 8d provides representative immunoblots for samples showing HSV-1 gB protein in comparison with GAPDH for HSV-1 infected HCEs at 24 hpi.

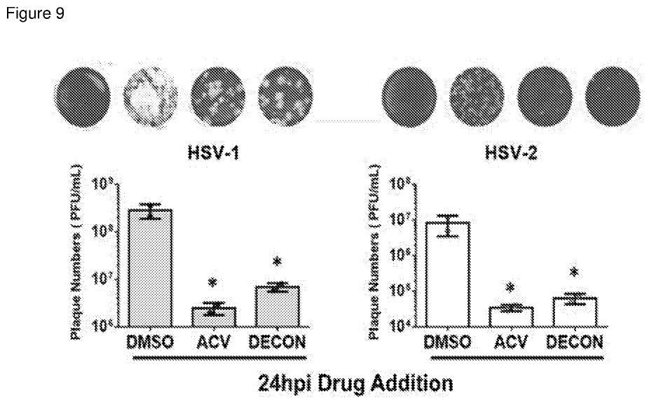

DECON addition was as effective as ACV alone even when the drug administration was carried out 24 post initial HSV infection (FIG. 9). HCEs or HFFs were infected with HSV-1 or HSV-2 at an MOI of 0.1 respectively for a period of 24 hours. 24 hours post infection, mock DMSO, 50 μM ACV or 0.1 mg/mL DECON were added to the infected cells. 24 hours post drug addition, the cells were collected, lysed and overlaid on Vero cells to conduct a plaque assay

Finally, the efficacy of DECON on other Herpesviridae viruses including HSV-2, pseudo rabies virus (PRV) and bovine herpes virus (BHV) was tested. Plaque assay data showing extent of virus inhibition by DECON in comparison to ACV, HPAC and mock DMSO for HSV-2, pseudo rabies virus (PRV) and bovine herpes virus (BHV) for intracellular virus collected 24 hpi is shown in FIGS. 8e, 8f and 8g. ACV loaded DECON was able to curb viral replication by multiple log 10 fold.

### Example 6

### Single Topical Dose of DECON on Alternate Days Restricts HSV-1 Replication In Vivo

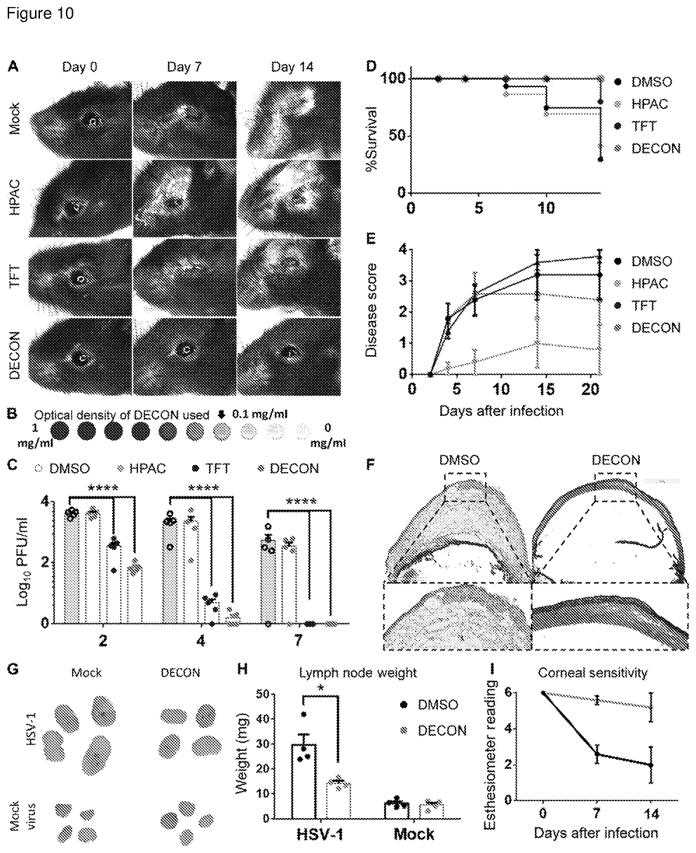

Next, we looked at the in vivo efficacy of DECON treatment in mice while also optimizing the dosing and administration. Since DECON sticks to the cells and releases drug upon viral infection, we hypothesized that when DECON is administered in vivo, alternate days of treatment would suffice to restrict viral replication. To compare the efficacy of DECON with existing topical antivirals, we used Trifluridine (TFT) as control along with HPAC and mock PBS-treated in a murine model of ocular infection. Treatments were started day 1 post infection and administered every alternate day for 11 days. The results showed that except for DECON, none of the other topical treatments were able to restrict viral replication (FIG. 10A). It is important to note that the concentration of DECON used in this experiment was as low as 0.1 mg/mL which had a low optical density shown in FIG. 10B. The reduction in viral replication was observed by plaque assays conducted using ocular washes collected on day 2, 4 and 7 post infection (FIG. 10C). Up to 20% of TFT, 60% of HPAC and 80% of mock treated mice succumbed to death by day 15 (FIG. 10D). Disease scores (0-4; 4 being severe) taken on days 2, 4, 7, 10, 14 and 21 were scored in a blinded fashion. DECON treatment was observed to be very effective where the mice showed no signs of blepharitis or corneal keratitis or any disease score during the period of infection (FIG. 10E).

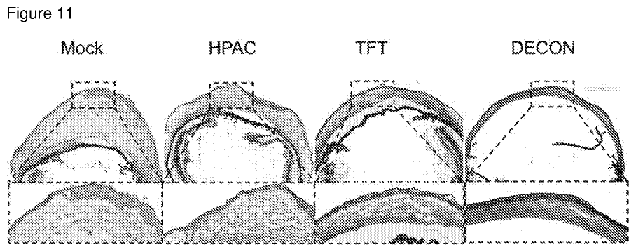

Mice were sacrificed on day 21 and their eyes were frozen in OCT medium for histology. 10 micron sections of the eye for all the groups were taken and stained with hematoxylin and eosin stain. It is pertinent to note that murine eyes treated with DECON were healthy and did not elicit an immune response when compared to those infected with HSV-1. (FIG. 10F and FIG. 11). Lymph nodes collected for HSV-1 or mock-infected mice treated with mock or DECON showed that DECON did not elicit any unnecessary immune response during or in the absence of infection (FIGS. 10G and 10H). Furthermore, no decline in the corneal sensitivity was observed for mice treated with DECON (FIG. 10I) as measured using a manual esthesiometer in mice. These results indicate that ACV delivery through our novel DECON platform can reduce the number of times these topical antivirals need to be administered.

### Example 7

### Intravaginal DECON Administration is as Effective as Systemic ACV Therapy During HSV-2 Infection

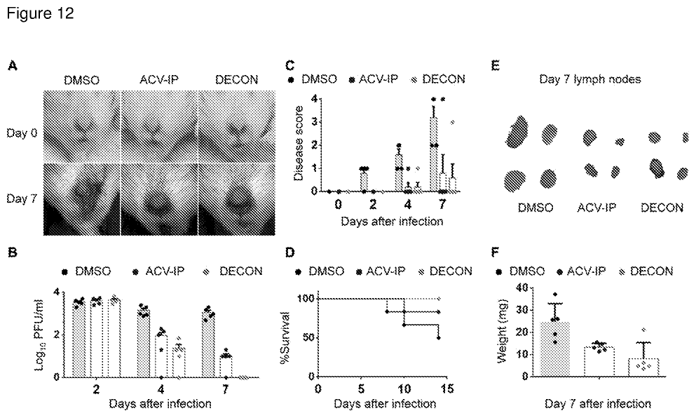

With the success of topical ocular therapy with DECON, it was investigated whether this delivery platform was effective for vaginal HSV-2 infections as well. Naïve Balb/c mice were primed with medroxyprogestrone 5 days prior to intravaginal HSV-2 infection. Starting at day 1 post infection, mice were either treated with topical DECON or systemic ACV (5 mg/kg) by intraperitoneal injections on alternate days. While systemic ACV was administered every day, DECON was administered intravaginally every. The genital area was imaged using a stereoscope on days 0 and 7 to visualize the extent of damage done to the site of infection. All the mice had equal amount of infection on day 2 post infection; however, a steady reduction in viral titers on day 4 and day 7 post infection for ACV and ACV loaded DECON treated mice was observed (FIG. 12B). This was observed by the phenotype of infection and analyzed by plaque assays from vaginal swabs. Furthermore, we observed only slight inflammation for the ACV and DECON treated mice compared to mock-treated mice which had severe inflammation and scarring of the vaginal and surrounding anogenital area (FIG. 12C). In the mock treated group, 60% of mice succumbed to death by day 14 post infection, while 20% of mice treated with systemic ACV and none of DECON treated mice succumbed to death (FIG. 5D). The lymph nodes collected for DECON and ACV treated mice day 14 post infection showed little to no increase in size, while mock treated mice had predominantly large and inflamed lymph nodes (FIGS. 12E and 12F). In conclusion, this confirms that alternate day treated with ACV loaded DECON is as beneficial as a daily dose of systemic ACV to curb HSV-2 infection in the genital region.

### Example 8

### Efficacy of DECON with Other Analogs of Acyclovir

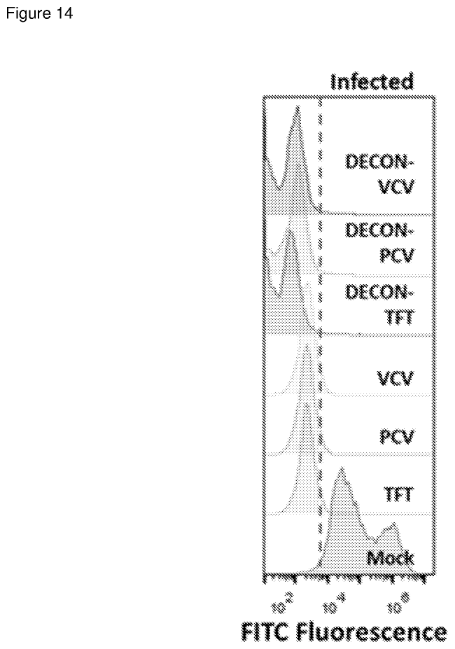

The following experiment was carried out to demonstrate that the DECON can successfully load other nucleoside analogs such as valaciclovir (VCV), penciclovir (PCV) and trifluridine (TFT). These nucleoside analogs were loaded as described in Example 4. These nucleoside analogs successfully loaded and demonstrated sustained release in a manner similar to ACV. FIG. 14 provides flow cytometry data demonstrating loading and sustained release. The red line indicates the demarcation between infected and non-infected cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

All documents referred to in this application are hereby incorporated by reference in their entirety.

What is claimed:

1. A composition comprising highly porous activated carbon (HPAC) and a therapeutic agent.

2. The composition of claim 1 wherein the HPAC is a microporous carbon.

3. The composition of claim 1 or 2 wherein the HPAC has a pore size ranging from about 10 Å to about 20 Å, or has a cumulative pore volume ranging from about 25 cc/g to about 0.75 cc/g or has a total pore volume ranging from 500 m²/g to about 3000 m²/g.

4. The composition of any one of claims 1-3 whereon the HPAC has a particle size distribution ranging from about 10 nm to about 500 μM.

5. The composition of any one of claim 1-4 wherein the therapeutic agent is an antibacterial agent, growth factor, cytokine, anticancer agent, cytotoxic agent, analgesic, anti-hypertension drug, anti-allergenic, an anti-seizure compound, non-steroidal anti-inflammatory drug, an antibiotic, growth hormone, parathyroid hormone, insulin, interferon, chemotherapeutic agent, immune suppression agents, antidepressant, antidiabetic agent, anti-parasitic, antidiarrheal agent, antimigraine agents, antipsychotics, antiparkinsonian agent, anxiolytic, or hypotensive agent. is an anti-viral agent, anan-diabetic agent, anti-inflammatory agent.

6. The composition of any one of claims 1-4 wherein the therapeutic agent is an anti-viral agent.

7. The composition of claim 6 wherein the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine or sofobuvir.

8. A composition of any one of claims 1-4, wherein the therapeutic agent is a live virus, live-attenuated virus or a fixed virion.

9. The composition of claim 8, wherein the virus, live-attenuated virus or fixed virion is Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

10. The composition of claim 8 or 9, wherein the composition further comprises an adjuvant.

11. A composition of any one of claims 1-10 further comprising a pharmaceutically acceptable carrier.

12. A drug delivery system comprising a therapeutic agent adsorbed to highly porous activated carbon (HPAC).

13. The drug delivery system of claim 12, wherein the therapeutic agent is delivered by parenteral, nasal, oral, pulmonary, topical, vaginal, or rectal administration.

14. The drug delivery system of claim 12 or 13, wherein therapeutic agent is delivered by sustained released.

15. The drug delivery system of any one of claims 12-14, wherein the therapeutic agent is delivered prophylactically or therapeutically.

16. The drug delivery system of any one of claims 12-15, wherein the HPAC is a microporous carbon.

17. The drug delivery system of any one of claims 12-16, wherein the HPAC has a pore size ranging from about 10 Å to about 20 Å, or has a cumulative pore volume ranging from 25 cc/g to about 0.75 cc/g or has a total pore volume ranging from 500 m²/g to about 3000 m²/g.

18. The drug delivery system of any one of claims 12-17, wherein the HPAC has a particle size distribution ranges from about 10 nM to about 500 μM.

19. The drug delivery system of any one of claims 12-18, wherein the therapeutic agent the is an antibacterial agent, growth factor, cytokine, anticancer agent, cytotoxic agent, analgesic, anti-hypertension drug, anti-allergenic, an anti-seizure compound, non-steroidal anti-inflammatory drug, an antibiotic, growth hormone, parathyroid hormone, insulin, interferon, chemotherapeutic agent, immune suppression agent, antidepressant, antidiabetic agent, anti-parasitic, antidiarrheal agent, antimigraine agent, antipsychotic, antiparkinsonian agent, anxiolytic, or hypotensive agent.

20. The drug delivery system of any one of claim 12-18, wherein the therapeutic agent is an anti-viral agent.

21. The drug delivery system of claim 20 wherein the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine or sofobuvir.

22. The drug delivery system of any one of claims 12-18, wherein the therapeutic agent is a live virus, live-attenuated virus or a fixed virion.

23. The drug delivery system of claim 22, wherein the virus, live-attenuated virus or fixed virion is Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

24. The drug delivery system of claim 22 or 23, wherein the composition further comprises an adjuvant.

25. A method of treating a viral infection comprising administering highly porous activated carbon (HPAC) in an amount that inhibits virus entry into a cell or reduces viral spread.

26. The method of claim 25, wherein the HPAC is a microporous carbon.

27. The method of claim 25 or 26, wherein the HPAC has a pore size ranging from about 10 Å to about 20 Å, or has a cumulative pore volume ranging from about 25 cc/g to about 0.75 cc/g or has a total pore volume ranging from about 500 m²/g to about 3000 m²/g.

28. The method of any one of claims 25-27, wherein the HPAC has a particle size distribution ranges from about 10 nM to about 500 μM.

29. The method of any one of claims 25-28, wherein an anti-viral agent is adsorbed within the HPAC.

30. The method of claim 29, wherein the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine or sofobuvir.

31. The method of any one of claims 25-30, wherein the infection is caused by Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

36. The method of claim 34, wherein the viral infection is a Herpes Simplex virus-1 or Herpes Simplex-2 infection.

37. The method of any one of claims 25-36, wherein the HPAC, composition or drug delivery system is administered by parenteral, nasal, oral, pulmonary, topical, vaginal, or rectal delivery.

38. The method of any one of claims 25-37, wherein the HPAC, composition or drug delivery system is administered by sustained released.

39. The method of any one of claims 25-38, wherein the HPAC, composition or drug is administered prophylactically or therapeutically.

40. A composition for treating a viral infection, wherein the composition comprises highly porous activated carbon (HPAC) in an amount that inhibits virus entry into a cell or reduces viral spread.

41. The composition of claim 40, wherein the HPAC is a microporous carbon.

42. The composition of claim 40 or 41, wherein the HPAC has a pore size ranging from about 10 Å to about 20 Å, or has a cumulative pore volume ranging from about 25 cc/g to about 0.75 cc/g or has a total pore volume ranging from about 500 m²/g to about 3000 m²/g.

43. The composition of any one of claims 40-42, wherein the HPAC has a particle size distribution ranges from 10 nM to 500 μM.

44. The composition of any one of claims 40-43, wherein an anti-viral agent is adsorbed within the HPAC.

45. The composition of claim 44, wherein the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine or sofobuvir.

46. The composition of any one of claims 40-45, wherein the infection is caused by Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

47. The composition of any one of claims 40-45, wherein the viral infection is a Herpes Simplex virus-1 or Herpes Simplex-2 infection.

48. A composition for treating a condition or disorder comprising a therapeutically effective amount of the composition of any one of claims 1-11 or the drug delivery system of any one of claims 12-24.

49. The composition of claim 48 wherein the condition or disorder is a viral infection.

50. The composition of claim 49, wherein the infection is caused by Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

51. The composition of claim 49, wherein the viral infection is a Herpes Simplex virus-1 or Herpes Simplex-2 infection.

52. The composition of any one of claims 40-51, wherein the HPAC, composition or drug delivery system is formulated for parenteral, nasal, oral, pulmonary, topical, vaginal, or rectal delivery.

53. The composition of any one of claims 40-51, wherein HPAC, composition or drug delivery system is administered by sustained released.

54. Use of highly porous activated carbon (HPAC) for the preparation of a medicament for the treatment of a viral infection, wherein the HPAC is in an amount that inhibits virus entry into a cell or reduces viral spread.

55. Use of claim 54, wherein the HPAC is a microporous carbon.

56. The use of claim 54 or 55, wherein the HPAC has a pore size ranging from about 10 Å to about 20 Å, or has a cumulative pore volume ranging from about 25 cc/g to about 0.75 cc/g or has a total pore volume ranging from about 500 m²/g to about 3000 m²/g.

57. The use of any one of claims 54-56, wherein the HPAC has a particle size distribution ranges from about 10 nM to about 500 μMX-X.

58. The use of any one of claims 54-57 wherein an anti-viral agent is adsorbed within the HPAC.

59. The use of claim 58, wherein the anti-viral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine or sofobuvir.

60. The use of any one of claims 54-59, wherein the infection is caused by Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

61. The use of any one of claims 54-59, wherein the viral infection is a Herpes Simplex virus-1 or Herpes Simplex-2 infection.

62. Use of a therapeutically effective amount of composition of any one of claims 1-11 or drug delivery system of any one of claims 12-24 for the preparation of a medicament for the treatment of a condition or disorder.

63. The use of claim 62, wherein the condition or disorder is a viral infection.

64. The use of claim 63, wherein the infection is caused Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

66. The use of claim 63, wherein the viral infection is a Herpes Simplex virus-1 or Herpes Simplex-2 infection.

67. The use of any one of claims 54-66, wherein the medicament is formulated for parenteral, nasal, oral, pulmonary, topical, vaginal, or rectal administration.

68. The use of any one of claims 54-67, wherein the medicament is formulated for sustained released.

69. The use of any one of claims 54-68, wherein the medicament is prophylactically administered or therapeutically administered.

70. A method of eliciting an immune response comprising administering a vaccine composition to a subject in need, wherein the vaccine composition comprises i) a live virus, a live-attenuated virus or a fixed virion, and ii) highly porous activated carbon (HPAC), wherein the vaccine elicits an immune response in the subject.

71. The method of claim 70, wherein the vaccine composition further comprises an adjuvant.

72. The method of claim 70 or 71, wherein the HPAC is a microporous carbon.

73. The method of any one of claims 70-72, wherein the HPAC has a pore size ranging from about 10 Å to about 20 Å, or has a cumulative pore volume ranging from 25 cc/g to about 0.75 cc/g or has a total pore volume ranging from 500 m²/g to about 3000 m²/g.

74. The method of any one of claims 70-73, wherein the HPAC has a particle size distribution ranges from about 10 nM to about 500 μM.

75. The method of any one of claims 70-74, wherein the virus, live-attenuated virus or fixed virion is Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

76. The method of any one of claims 70-75 wherein the vaccine composition is administered by oral, nasal, vaginal, rectal, ocular or sublingual route.

77. A composition for eliciting an immune response comprising i) a live virus, a live-attenuated virus or a fixed virion, and ii) highly porous activated carbon (HPAC).

78. The composition of claim 77, further comprises an adjuvant.

79. The composition of claim 77 or 78, wherein the HPAC is a microporous carbon.

80. The composition of any one of claims 77-79, wherein the HPAC has a pore size ranging from about 10 Å to about 20 Å, or has a cumulative pore volume ranging from 25 cc/g to about 0.75 cc/g or has a total pore volume ranging from 500 m²/g to about 3000 m²/g.

81. The composition of any one of claims 77-80, wherein the HPAC has a particle size distribution ranges from about 10 nM to about 500 μM.

82. The composition of any one of claims 77-81, wherein the virus, live-attenuated virus or fixed viron is Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

83. The composition of any one of claims 77-81, wherein composition formulated for administration by oral, nasal, vaginal, rectal, ocular or sublingual route.

84. Use of a vaccine composition for the preparation of a medicament for eliciting an immune response, wherein the vaccine composition comprises i) a live virus, a live-attenuated virus or a fixed virion, and ii) highly porous activated carbon (HPAC), wherein the vaccine elicits an immune response in a subject.

85. The use of claim 84, wherein the vaccine composition further comprises an adjuvant.

86. The use of claim 84 or 85, wherein the HPAC is a microporous carbon.

87. The use of any one of claims 84-86, wherein the HPAC has a pore size ranging from about 10 Å to about 20 Å, or has a cumulative pore volume ranging from 25 cc/g to about 0.75 cc/g or has a total pore volume ranging from 500 m²/g to about 3000 m²/g.

88. The use of any one of claims 84-87, wherein the HPAC has a particle size distribution ranges from about 10 nM to about 500 μM.

89. The use of any one of claims 84-88, wherein the virus, live-attenuated virus or fixed viron is Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

90. The use of any one of claims 84-89, wherein the medicament is formulated for administration by oral, nasal, vaginal, rectal, ocular or sublingual routes.


SEQUENCE LISTING (8 sequences)

SEQ ID NO: 1 - DNA, 18 bp, Artificial Sequence
  Synthetic Polynucleotide
tccactggcgtcttcacc

SEQ ID NO: 2 - DNA, 21 bp, Artificial Sequence
  Synthetic Polynucleotide
ggcagagatgatgaccctttt

SEQ ID NO: 3 - DNA, 21 bp, Artificial Sequence
  Synthetic Polynucleotide
gatggcaaccagttccagaag

SEQ ID NO: 4 - DNA, 24 bp, Artificial Sequence
  Synthetic Polynucleotide
aaagaggttgaagatctgctggat

SEQ ID NO: 5 - DNA, 21 bp, Artificial Sequence
  Synthetic Polynucleotide
ctccactacagctctttccat

SEQ ID NO: 6 - DNA, 21 bp, Artificial Sequence
  Synthetic Polynucleotide
gtcaaagttcatcctgtcctt

SEQ ID NO: 7 - DNA, 21 bp, Artificial Sequence
  Synthetic Polynucleotide
agcccatgttgtagcaaaccc

SEQ ID NO: 8 - DNA, 21 bp, Artificial Sequence
  Synthetic Polynucleotide
ggacctgggagtagatgaggt